(19) United States
(12) Patent Application Publication
(10) Pub. No.: US 2019/0216831 A1
(43) Pub. Date: Jul. 18, 2019

(54) ANTIVIRAL COMPOSITION

(71) Applicant: EQUIGERMINAL SA, Cantanhede (PT)

(72) Inventors: Isabel Maria Fidalgo dos Santos Silva Carvalho, Coimbra (PT); Alexandre Simao Vieira Pires, Coimbra (PT)

(73) Assignee: EQUIGERMINAL SA, Cantanhede (PT)

(21) Appl. No.: 15/574,155

(22) PCT Filed: May 31, 2016
(86) PCT No.: PCT/PT2016/050012

§ 371 Date: Nov. 14, 2017

(30) Foreign Application Priority Data
Jun. 1, 2015 (GB) 1509431.1

(51) Int. Cl. A61K 31/675; A61P 31/22

(52) U.S. Cl. CPC A61K 31/675; A61P 31/22

(57) ABSTRACT

The present invention relates to antiviral compositions. The present invention also relates to compositions for use in the therapy of equine viral infections. In particular, the present invention relates to compositions comprising at least one anti-viral compound for use in a method of therapy of an equine viral infection and/or infection by an equine virus in an animal.

### FIELD OF THE INVENTION

The present invention relates generally to the field of medicine. Further, various embodiments relate to pharmaceutical medicine. In particular, the invention relates to compositions for use in the therapy of equine viral infections.

### BACKGROUND OF THE INVENTION

Equine infectious anemia (EIA) or swamp fever is an ancient lentiviral disease of equines and equids. The EIAV (Equine infectious anemia virus) retrovirus is known, historically, to be the first viral agent responsible for an animal disease, the Swamp fever in horses.

The disease was first reported in Europe, in France, in 1843 (Lignee, 1843). At present, it is considered to be distributed worldwide. In recent years, several clinical cases of swamp fever were reported by the World Organization for Animal Health (OIE) in the European horse population.

The ancient origin of this virus, geographical dispersion, persistence over time in countries with monitoring plans, and the ability of EIAV to be highly variable suggest that much remains to be leamed about this virus. Means and methods for diagnosis and treatment are thus needed.

Currently, there is no known cure for the disease. Diagnosis, quarantine/isolation or elimination of seropositive animals is the only way to control the disease. The current immunodiagnostic tests for the detection of anti-EIAV antibodies in the serum of horses infected with EIAV utilize the whole virus as an antigen, or viral recombinant proteins.

The OIE official test to diagnose the presence of EIA has been the presence of antibodies specific for the disease in the serum of affected animals using the Coggins or agar gel diffusion test (AGID), as described in U.S. Pat. Nos. 3,929,982 and 3,932,601.

Recently, Fidalgo-Carvalho and colleagues have identified a new and previously uncharacterised virus, which was obtained from horses with discordant results for EIAV testing i.e. the horses were positive for EIAV in an immunoblot but negative for EIAV in both the AGID test and an ELISA. This new uncharacterised virus was named New Equine Virus (NEV), as described in PCT/PT2014/000077.

Adefovir (including its pro-drug form, adefovir dipivoxil) is a nucleoside and reverse transcription inhibitor that is currently used as a prescription medicine to treat human Hepatitis B virus (HBV) infections. It may also be used to treat human herpes simplex virus infections. Adefovir is a failed treatment for HIV infections.

Adefovir was first made at the Institute of Organic Chemistry and Biochemistry, Academy of Sciences of the Czech Republic by Antonin Holy, and the drug was developed by Gilead Sciences for HIV with the brand name Preveon. However, the drug did not receive FDA approval for HIV, due to concerns about the severity and frequency of kidney toxicity when dosed at 60 or 120 mg. Adefovir is effective against human hepatitis B (HBV) infections, with a much lower dose of 10 mg. Adefovir is now sold for this indicatbn under the brand name Hepsera.

Adefovir works by blocking reverse transcriptase, an enzyme crucial for the HBV to reproduce in the body. It is approved for the treatment of chronic hepatitis B in adults with evidence of active viral replication and either evidence of persistent elevations in serum aminotransferases (primarily ALT) or histologically active disease.

Adefovir dipivoxil contains two pivaloyloxymethyl units, making it a pro-drug form of adefovir. A pro-drug form of adefovir was previously called bis-POM PMEA.

The structural formula of adefovir is as follows:

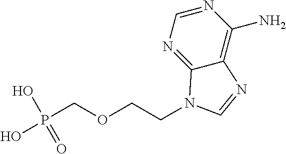 [SMILES: Nc1ncnc2c1ncn2CCOCP(=O)(O)O]

The chemical name of adefovir dipivoxil is 9-[2-[[bis[(pivaloyloxy)methoxy]phosphinyl]-methoxy]ethyl]adenine. It has a molecular formula of C₂₀H₃₂N₅O₈P, a molecular weight of 501.48 g/mol, and a structural formula as follows:

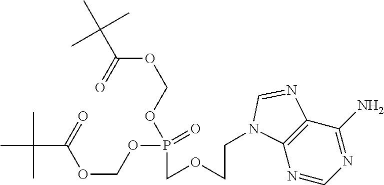 [SMILES: CC(C)(C)C(=O)OCOP(=O)(COCCn1cnc2c(N)ncnc21)OCOC(=O)C(C)(C)C]

Tenofovir is another nucleotide analogue reverse transcriptase inhibitors (NRTI). Tenofovir is marketed by Gilead Sciences under the trade name Viread (as the disoproxil fumarate pro-drug/salt, or TDF). TDF (Viread) is FDA-approved for the treatment of HIV and chronic hepatitis B. It is also marketed under the brand name Reviro. Tenofovir is also available in a fixed-dose combination with emtricitabine in a product with the brand name Truvada for once-a-day dosing. Atripla, a fixed-dose triple combination of tenofovir, emtricitabine, and efavirenz, was approved by the FDA as a single daily dose for the treatment of HIV.

Tenofovir is indicated in combination with other antiretroviral agents for the teatment of HIV-1 infection in human adults and human pediatric patients 2 years of age and older. Tenofovir is also indicated for the treatment of chronic hepatitis B in human adults and human pediatric patients 12 years of age and older. It has also been found that both tenofovir alone and a tenofovir/emtricitabine combination significantly decreased the risk of contracting HIV.

Tenofovir was initially synthesized by Antonin Holy at the Institute of Organic Chemistry and Biochemistry, Academy of Sciences of the Czech Republic in Prague, as described in U.S. Pat. No. 4,808,716. In 1997 researchers from Gilead and the University of California, San Francisco demonstrated that tenofovir exhibits anti-HIV effects in humans when dosed by subcutaneous injection. A medicinal chemistry team at Gilead further developed a modified version of tenofovir, tenofovir disoproxil fumarate, for oral delivery.

The IUPAC name of tenofovir is [(2R)-1-(6-aminopurin-9-yl)propan-2-yl]oxymethylphosphonic acid and its structural formula is as follows:

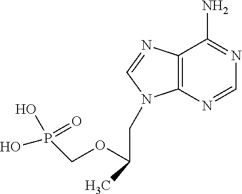 [SMILES: C[C@H](Cn1cnc2c(N)ncnc21)OCP(=O)(O)O]

The structural formula of the tenofovir disoproxil pro-drug (shown as the fumarate salt, TDF) is as follows:

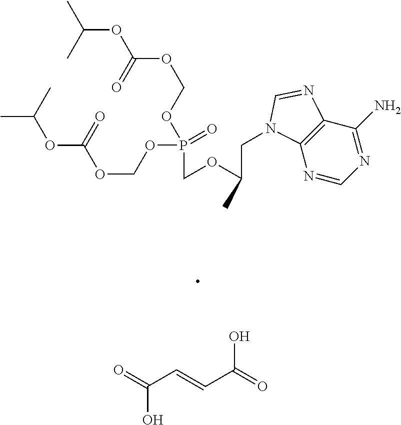 [SMILES: CC(C)OC(=O)OCOP(=O)(CO[C@H](C)Cn1cnc2c(N)ncnc21)OCOC(=O)OC(C)C ; O=C(O)/C=C/C(=O)O]

The structural formula of the tenofovir alafenamide pro-drug (shown as the fumarate salt, TAF) is as follows:

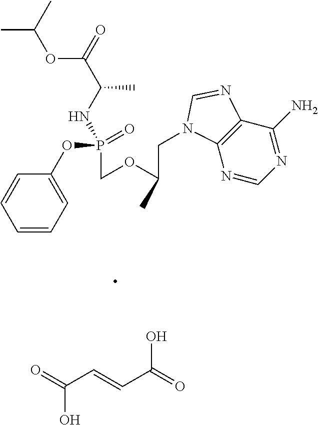 [SMILES: CC(C)OC(=O)[C@H](C)N[P@](=O)(CO[C@H](C)Cn1cnc2c(N)ncnc21)Oc1ccccc1 ; O=C(O)/C=C/C(=O)O]

U.S. Pat. No. 5,663,159 describes the synthesis of anti-virally active pro-drugs of phosphonate nucleotide analogs. Examples of such active pro-drugs include adefovir dipivoxil and tenofovir disoproxil.

U.S. Pat. No. 6,451,340 describes crystalline forms of adefovir dipivoxil and methods of preparing the same.

Hence, there exists a need for pharmaceutical treatments of equine viral diseases and infections.

### STATEMENT OF INVENTION

The present invention provides a composition comprising at least one anti-viral compound (for example for some embodiments an anti-retroviral compound) for use in a method of therapy of an equine viral infection and/or infection by an equine virus in an animal.

The present invention also provides a method of therapy of an equine viral infection and/or infection by an equine virus in an animal comprising administering to said animal a composition comprising at least one anti-viral compound (for example an anti-retroviral compound).

Advantageously, the compositions for use according to the present invention are capable of treating, preventing or diagnosing an equine viral infection. Advantageously, this means that more animals (e.g. horses) with equine viral infections infection (i.e. seropositive animals) can be treated, and the spread of infection can be limited.

In a further aspect, the present invention provides a diagnostic method comprising:
(a) obtaining a sample from an animal;
(b) admixing with said sample a composition comprising at least one anti-viral compound (for example an anti-retroviral compound);
(c) determining the presence or absence of an equine virus and/or equine viral particles and/or equine viral peptides and/or equine viral nucleic acids in said sample.

The present invention also provides a method of screening for an equine virus, said method comprising:
(a) obtaining a sample from an animal;
(b) admixing with said sample a composition comprising an anti-viral compound (for example an anti-retroviral compound);
(c) determining the presence or absence of an equine virus and/or equine viral particles and/or equine viral peptides and/or equine viral nucleic acids in said sample.

In another aspect, the present invention provides a method for controlling a viral infection in a group of animals comprising the identification of a viral infection and, optionally, the isolation of an equine virus-infected animal from other animals.

In related aspects, the present invention provides a pharmaceutical composition comprising at least one anti-viral compound (for example an anti-retroviral compound) for use according to the invention and a pharmaceutically acceptable carrier, vehicle, diluent or excipient.

Also provided by the present invention is a kit comprising at least one anti-viral compound (for example an anti-retroviral compound) for use according to the invention and optionally instructions for administration to said animal.

In a further aspect, compositions according to the present invention may be used for modulating reverse transcriptase activity in an equine virus; for inhibiting the replication of an equine virus in vitro, and/or for promoting the survival of animal cell infected with an equine virus in vitro.

### BRIEF DESCRIPTION OF FIGURES

FIG. 1. NEV morphology analysed by electron microscopy. Negative staining of viral particles (panel A, B and C) and staining of infected cells (panel D).

FIG. 2A. Cell viability of equine dermal (ED) cells at 11 days post NEV infection, analysed using the Presto blue cell viability assay. The differences observed between NEV-infected treated and untreated cells were statistically significant by using 2-way ANOVA and Bonferroni post tests with a p value <0.0001.

FIG. 2B. Dose response curve of cell viability of NEV infected cells in the presence of ten serial dilutions (1:2) of adefovir dipivoxil with concentrations ranging from 5 to 2560 nM. The results were analysed by Prism software by using the four-parameter function (log (drug) vs. response assuming a variable slope).

FIG. 3A. Cell viability of MacF cells at 11 days post NEV infection, analysed using the Presto blue cell viability assay. The differences observed between NEV-infected treated and untreated cells were statistically significant by using 2-way ANOVA and Bonferroni post tests with a p value <0.0001.

FIG. 3B. Dose response curve of cell viability of macrophage cell lines infected with NEV in the presence of eleven serial dilutions (1:4) of adefovir dipivoxil with concentrations ranging from 0.05 nM to 28 μM. The results were analysed by Prism software by using the four-parameter function (log (drug) vs. response assuming a variable slope).

FIG. 4A. Adefovir dipivoxil possess antiviral activity against EIAV (I). EIAV-infected equine dermal cells were treated with either adefovir, tenofovir, nevirapine or zidovudine at 1 or 10 μM for up to 20 days post-infection. Samples were screened for number of viral particles/mL of cell culture supernatant using RT qPCR techniques.

FIG. 4B. Adefovir dipivoxil possess antiviral activity against EIAV (II). EIAV-infected Equine Dermal cells were treated with either darunavir, indinavir, daclatasvir or cyclosporin A at 1 or 10 μM for up to 20 days post-infection. Samples were screened for number of viral particles/mL of cell culture supernatant using RT qPCR techniques.

FIG. 4C. The effect of adefovir dipivoxil on EIA_{WYO} viral replication in ED cells. The results were analysed by Prism software using the four-parameter function (log (drug) vs. response assuming a variable slope) to calculate an IC₅₀.

FIG. 5A. Dose response curve of cell viability in the presence of different concentrations of adefovir dipivoxil. Confluent ED cell monolayers were washed twice with HBSS to remove non-adherent cells and pre-treated with adefovir dipivoxil for 6 days. To determine drug CC₅₀, twelve different concentrations of 4, 6, 9, 13.5, 20.250, 30.370, 45.560, 68.340, 102.520, 153.770, 230.660 and 2000 μM were performed in quintuplicates per each drug concentration. Cell viability was assessed by using the PrestoBlue reagent and incubated for 24 hours. The results were analysed by Prism software by using the four-parameter function (log (drug) vs. response assuming a variable slope).

FIG. 5B. Dose response curve of cell viability in the presence of different concentrations of adefovir dipivoxil. Confluent MacF cell monolayers were washed twice with HBSS to remove non-adherent cells and pre-treated with adefovir dipivoxil for 7 days. To determine drug CC₅₀, six different concentrations of 45.560, 68.340, 102.520, 153.770, 230.660 and 2000 μM were performed in quintuplicates per each drug concentration. Cell viability was assessed by using the PrestoBlue reagent and incubated for 24 hours. The results were analysed by Prism software by using the four-parameter function (log (drug) vs. response assuming a variable slope).

FIG. 6A. Adefovir dipivoxil IC₅₀ for EHV-1 obtained by qPCR in ED cells. Viral particle production was assayed at day 3 post infection in cell culture supernatants in triplicates and determined by means of qPCR. The results were analysed by Prism software using the four-parameter function (log (drug) vs. response assuming a variable slope) to calculate an IC₅₀.

FIG. 6B. Adefovir dipivoxil IC₅₀ for EHV-1 obtained by cell viability assays in ED cells. To determine drug IC₅₀ for EHV-1 in ED cell viability was assessed by using the PrestoBlue reagent and incubated for 24 hours. EHV-1 infected ED cell monolayers treated with different concentrations of adefovir dipivoxil were assayed at 6 days post infection. The results were analysed by Prism software by using the four-parameter function (log (drug) vs. response assuming a variable slope).

FIG. 7A. Adefovir dipivoxil IC₅₀ for EHV-1 obtained by qPCR in Macrophage-like cell lines. Viral particle production was assayed at day 3 post infection in cell culture supernatants in triplicates and determined by means of qPCR. The results were analysed by Prism software using the four-parameter function (log (drug) vs. response assuming a variable slope) to calculate an IC₅₀.

FIG. 7B. Adefovir dipivoxil IC₅₀ for EHV-1 obtained by cell viability assays in Macrophage-like cell lines. To determine drug IC₅₀ for EHV-1 in MacF Cell viability was assessed by using the PrestoBlue reagent and incubated for 24 hours. EHV-1 infected MacF cell monolayers treated with different concentrations of adefovir dipivoxil were assayed at 6 days post infection. The results were analysed by Prism software by using the four-parameter function (log (drug) vs. response assuming a variable slope).

FIG. 8A. Tenofovir disoproxil fumarate IC₅₀ for EHV-1 obtained by qPCR in ED cells. Viral particle production was assayed at day 3 post infection in cell culture supernatants in triplicates and determined by means of qPCR. The results were analysed by Prism software using the four-parameter function (log (drug) vs. response assuming a variable slope) to calculate an IC₅₀.

FIG. 8B. Tenofovir disoproxil fumarate IC₅₀ for EHV-1 obtained by cell viability assays in ED cells. To determine drug IC₅₀ for EHV-1 in ED cell viability was assessed by using the PrestoBlue reagent and incubated for 24 hours. EHV-1 infected ED cell monolayers treated with different concentrations of Tenofovir disoproxil fumarate were assayed at 6 days post infection. The results were analysed by Prism software by using the four-parameter function (log (drug) vs. response assuming a variable slope).

FIG. 9. Dose response curve of cell viability in the presence of different concentrations of Tenofovir disoproxil fumarate. Confluent ED cell monolayers were washed twice with HBSS to remove non-adherent cells and pre-treated with tenofovir disoproxil fumarate for 3 days. To determine drug CC₅₀, 1 μM and ten 1:1.3 serial dilutions from 25.39 to 350 μM and were performed in quintuplicates per each drug concentration. Cell viability was assessed by using the PrestoBlue reagent and incubated for 24 hours. The results were analysed by Prism software by using the four-parameter function (log (drug) vs. response assuming a variable slope).

FIG. 10A. Tenofovir disoproxil fumarate IC₅₀ for EHV-1 obtained by qPCR in Macrophage-like cell lines. Viral particle production was assayed at day 3 post infection in cell culture supernatants in triplicates and determined by means of qPCR. The results were analysed by Prism software using the four-parameter function (log (drug) vs. response assuming a variable slope) to calculate an IC₅₀.

FIG. 10B. Tenofovir disoproxil fumarate IC₅₀ for EHV-1 obtained by cell viability assays in Macrophage-like cell lines. To determine drug IC₅₀ for EHV-1 in MacF Cell viability was assessed by using the PrestoBlue reagent and incubated for 24 hours. EHV-1 infected MacF cell monolayers treated with different concentrations of Tenofovir disoproxil fumarate were assayed at 6 days post infection. The results were analysed by Prism software by using the four-parameter function (log (drug) vs. response assuming a variable slope).

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

### Anti-Viral Compound

A first aspect of the present invention relates to a composition comprising at least one anti-viral compound for use in a method of therapy of an equine viral infection and/or infection by an equine virus in an animal.

In all aspects and embodiments herein, the anti-viral compound of the invention may be an anti-retroviral compound.

An “anti-viral compound” is intended to encompass any compound which is known to mitigate (or is capable of mitigating) the normal biological activity or infectious capability of a virus, whether in vitro or in vivo, whether directly or indirectly. For example, the substance may prevent replication of the virus and/or may prevent the reverse transcription of retroviral nucleic acids and/or may inhibit viral protease activity and/or may prevent the onset or progression of a viral infection in an animal or host.

An “anti-retroviral compound” is intended to encompass any compound which is known to mitigate (or is capable of mitigating) the normal biological activity or infectious capability of a retrovirus, whether in vitro or in vivo, whether directly or indirectly. For example, the substance may prevent replication of the retrovirus and/or may prevent the reverse transcription of retroviral nucleic acids and/or may inhibit retroviral protease activity and/or may prevent the onset or progression of a retroviral infection in an animal or host.

In one embodiment, the anti-viral compound is a phosphonate nucleotide, or a pro-drug, equivalent or derivative thereof.

In one embodiment, the anti-viral compound is selected from adefovir or a pro-drug, equivalent or derivative thereof; and/or tenofovir or a pro-drug, equivalent or derivative thereof.

The composition of the present invention may comprise a combination of two or more of adefovir or a pro-drug, equivalent or derivative thereof; and tenofovir or a pro-drug, equivalent or derivative thereof.

In a preferred embodiment, the composition of the present invention comprises one or more of adefovir dipivoxil, tenofovir disoproxil, tenofovir disproxil fumarate and/or tenofovir alafenamide.

In a preferred embodiment, the composition of the present invention comprises adefovir dipivoxil.

In a preferred embodiment, the composition of the present invention comprises tenofovir disoproxil.

In another embodiment, the anti-viral compound is a compound of the Formula (I):

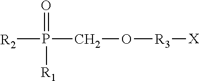

wherein
X is adenine, guanine, cytosine, thymine, uracil, 2,6-diaminopurine or hypoxanthine; R₁ and R₂ are the same or different and are each independently selected from the group consisting of: OR₄, NH₂, NHR₄, NHR₅, NHR₄R₅, or N(R₅)₂; in some cases, R₁ and R₂ are linked with each other to form a cyclic group, in other cases, R₁ or R₂ is linked to R₃ to form a cyclic group;
R₃ represents C₁-C₂₀ alkyl which may be unsubstituted or substituted by substituents independently selected from the group consisting of hydroxy, oxygen, nitrogen and halogen; when R₃ is CH(CH₂OR₆)CH₂, R₁ and R₂ each independently represent OH, and R₆ is a hydrolyzable ester group;
R₄ represents hydrogen or a physiologically hydrolyzable group; R₄ may also be R₅′;
R₅ represents C₁-C₂₀ alkyl, alkoxy, amino, aryl or aryl-alkyl which may be substituted or unsubstituted by substitutents independently selected from the group consisting of hydroxyl, oxygen, nitrogen and halogen;
R₅′ represents C₄-C₂₀ alkyl, aryl or aryl-alkyl which may be substituted or unsubstituted by substitutents independently selected from the group consisting of hydroxyl, oxygen, nitrogen and halogen;
or a pharmaceutical or veterinary acceptable salt thereof.

In some embodiments, the physiologically hydrolyzable group may be an ester, carbonate or carbamate group.

In some embodiments, the physiologically hydrolyzable group is selected from the group consisting of CH₂C(O)N(R₅)₂, CH₂C(O)OR₅, CH₂OC(O)R₅, CH(R₅)OC(O)R₅ (R, S or RS stereochemistry), CH(R₅)C(O)R₅ (R, S or RS stereochemistry), CH₂C(R₅)₂CH₂OH or CH₂OR₅.

In one particular embodiment, R₃ is ethyl.

In another particular embodiment, R₅ is selected from: tert-butyl and OCH(CH₃)₂.

In another particular embodiment, R₅′ is phenyl.

In another embodiment, R₄ may also be R₅′ provided that R₄ and R₅′ are not simultaneously alkyl.

In another embodiment, neither of R₄ or R₅′ is (CH₂)₃O(CH₂)₁₅CH₃.

In one embodiment, the anti-viral compound of the present invention has the general structural formula as shown in Formula (II):

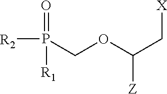

wherein
X, R₁ and R₂ are as described in Formula (I);
Z represents hydrogen, methyl, CH₂OR₆(R, S or RS stereochemistry), hydroxymethyl, or substituted or unsubstituted lower alkyl; when Z is CH₂OR₆, R₁ and R₂ may additionally be independently chosen from OH; and
R₆ represents a hydrolyzable group;
or a pharmaceutical or veterinary acceptable salt thereof.

In some embodiments, when Z is CH₂OR₆, R₆ is not CH₂Ph, and R₁ and R₂ are not both ethoxy. In a further embodiment, when R₁ is methoxy and R₂ is hydrogen, R₆ is not methyl. In a further embodiment, when R₁ is methoxy and R₂ is hydrogen, R₆ is not octyl.

In another embodiment, the anti-viral compound of the present invention has the general structural formula as shown in Formula (III):

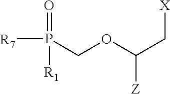

wherein
X and R₁ are as previously described in Formula (I);
Z is as described in Formula (II);
R₇ represents OH, NH₂, NHR₅ or N(R₅)₂; and
R₅ is as described in Formula (I);
or a pharmaceutical or veterinary acceptable salt thereof.

The compound of Formula (II) may be stereoisomerically pure, such as a stereoisomerically pure compound of the Formula (IV) shown below:

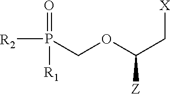

The compound of Formula (III) may be stereoisomerically pure, such as a stereoisomerically pure compound of the Formula (V) shown below:

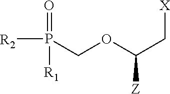

The anti-viral compound according to the present invention may be selected from adefovir and/or tenofovir.

Anti-viral compounds according to the present invention, and in particular compounds of the Formulae (I)-(V) may be synthesized according to the reaction schemes set out in U.S. Pat. No. 5,663,159.

“Alkyl” means a saturated hydrocarbon radical having a number of carbon atoms, for example 1 to 20 carbon atoms, preferably 1 to 10 carbon atoms, more preferably 1 to 5 carbon atoms, most preferably 1 to 3 carbon atoms, that may be branched or unbranched. Nonlimiting examples of alkyl radicals include methyl, ethyl, n-propyl, isopropyl, n-butyl, isobutyl, sec-butyl, tert-butyl, tert-amyl, pentyl, hexyl, heptyl, octyl and the like, wherein methyl, ethyl, n-propyl, and isopropyl represent specifically preferred examples.

A “lower alkyl” is a shorter alkyl, e.g., one containing from one to about six carbon atoms. Also, as referred to herein, a “lower” alkyl, alkenyl or alkynyl moiety (e.g., “lower alkyl”) is a chain comprised of 1 to 10, preferably from 1 to 8, carbon atoms in the case of alkyl and 2 to 10, preferably 2 to 8, carbon atoms in the case of alkene and alkyne.

The term “C₁ to C₂₀ alkyl” as used herein and in the claims (unless the context indicates otherwise) means saturated or unsaturated, branched or straight chain hydrocarbon group having 1 to 20 carbon atoms such as methyl, ethyl, propyl, isopropyl, butyl, isobutyl, t-butyl, etc. Unless otherwise specified in the particular instance, the term “substituted or unsubstituted” as used herein and in the claims is intended to mean hydrocarbon group wherein an atom, element or group is regarded as having replaced a hydrogen atom. Said substituted alkyl groups are preferably substituted with a member selected from the group consisting of hydroxyl, oxygen, nitrogen and halogen.

“Alkoxy” means an oxygen radical having a hydrocarbon chain substituent, where the hydrocarbon chain is an alkyl or alkenyl (i.e., —O-alkyl or —O-alkenyl). Examples of alkoxy radicals include methoxy, ethoxy, n-propoxy, isopropoxy, n-butoxy, isobutoxy, sec-butoxy, tert-butoxy, allyloxy and the like.

“Aryl” is an aromatic hydrocarbon ring. Aryl rings are monocyclic or fused bicyclic ring systems. Monocyclic aryl rings contain 6 carbon atoms in the ring. Monocyclic aryl rings are also referred to as phenyl rings. Bicyclic aryl rings contain from 8 to 17 carbon atoms, preferably 9 to 12 carbon atoms, in the ring. Bicyclic aryl rings include ring systems wherein one ring is aryl and the other ring is aryl, cycloalkyl, or heterocycloalkyl. Preferred bicyclic aryl rings comprise 5-, 6- or 7-membered rings fused to 5-, 6-, or 7-membered rings. Aryl rings may be unsubstituted or substituted with from 1 to 4 substituents on the ring. Aryl may be substituted with halo, cyano, nitro, hydroxy, carboxy, amino, acyl, amino, alkyl, heteroalkyl, haloalkyl, phenyl, aryloxy. alkoxy, heteroalkyloxy, carbamyl, haloalkyl, methylenedioxy, heteroaryloxy, or any combination thereof. Examples of aryl rings include naphthyl, tolyl, xylyl, and phenyl.

“Halo” or “halogen” may be fluoro, chloro, bromo or iodo.

By “physiologically hydrolyzable ester group” it is meant an ester bond or link which may be cleaved as a result of a physical, chemical or biological process in a living organism. An example of such a group is a diester-phosphonate link to nucleoside analogs of pyrimidine and purine bases.

The term “active ingredient” as used herein encompasses one or more compounds according to the present invention or isomers, solvates, pharmaceutical or veterinary acceptable salts or metabolites thereof.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question. Consequently, if a mixture of enantiomers is obtained during any of the preparation methods described herein, it can be separated by liquid chromatography using a suitable chiral stationary phase. Suitable chiral stationary phases are, for example, polysaccharides, in particular cellulose or amylose derivatives. Commercially available polysaccharide based chiral stationary phases are ChiralCel™ CA, OA, OB, OC, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like. The terms cis and trans are used herein in accordance with Chemical Abstracts nomenclature and refer to the position of the substituents on a ring moiety. The absolute stereochemical configuration of the compounds of formula may easily be determined by those skilled in the art while using well-known methods such as, for example, X-ray diffraction.

Those of skill in the art will also recognize that the compounds of the invention may exist in many different protonation states, depending on, among other things, the pH of their environment. While the structural formulae provided herein depict the compound in only one of several possible protonation states, it will be understood that these structures are illustrative only, and that the invention is not limited to any particular protonation state, any and all protonated forms of the compounds are intended to fall within the scope of the invention.

### Pro-Drug, Equivalent, Derivative

The present invention provides any suitable pro-drug, equivalent or derivative of the compounds described herein.

The pro-drug, derivative or equivalent may be selected from adefovir dipivoxil and/or tenofovir disoproxil and/or tenofovir disproxil fumarate and/or tenofovir alafenamide.

The term “pro-drug” as used herein and in the claims (unless the context indicates otherwise) denotes a derivative of an active drug which is converted after administration back to the active drug. More particularly, it refers to derivatives of nucleotide phosphonate antiviral drugs which are capable of undergoing hydrolysis of a physiologically hydrolysable group, such as an ester moiety or oxidative cleavage of the ester or amide moiety so as to release active free drug. The physiologically hydrolyzable groups serve as pro-drugs by being hydrolyzed in the body to yield the parent drug per se.

Embodiments of this invention relate to various precursor or “pro-drug” forms of the compounds of the present invention. It may be desirable to formulate the compounds of the present invention in the form of a chemical species which itself is not significantly biologically-active, but which when delivered to the animal will undergo a chemical reaction catalyzed by the normal function of the body of the animal, inter alia, enzymes present in the stomach or in blood serum, said chemical reaction having the effect of releasing a compound as defined herein. The term “pro-drug” thus relates to these species which are converted in vivo into the active pharmaceutical ingredient.

The pro-drugs of the present invention can have any form suitable to the formulation, for example, esters, more specifically alkylesters, are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolyzable precursor, inter alia an ester, an amide, and the like, may be used. The counterpart of the active pharmaceutical ingredient in the pro-drug can have different structures such as an amino acid or peptide structure, alkyl chains, sugar moieties.

The terms “equivalent” and “derivative” are intended to encompass any structural, isomeric, enantiomeric or diastereomeric derivative of the compounds described herein (e.g. by addition of one or more functional or non-functional groups) having an equivalent function to the compounds described herein. The activity of the equivalent or derivative may be greater or lesser than that of the compounds described herein. The terms are also intended to cover salts, solvates and metabolites of the compounds described herein, such as pharmaceutically or veterinary acceptable salts thereof. The terms are also intended to cover different forms of the compounds described herein, such as a crystalline form. The terms are also intended to cover different isomers of the compounds described herein, such as R-, S-, or R-S stereoisomers.

The term “isomers” as used herein means all possible isomeric forms, including tautomeric forms, which the compounds of the invention may possess. Unless otherwise stated, the standard chemical designation refers to all possible stereochemically isomeric forms, including all diastereomers and enantiomeres (since the compounds of the invention may have at least one chiral center) of the basic molecular structure. More particularly, unless otherwise stated, stereogenic centres may have either the R- or S-configuration, and substituents may have either cis- or trans-configuration.

In particular embodiments, the pro-drugs of the compounds of the present invention—for example the compounds of any one of Formulae (I)-(V)—are characterized by modified R₄ groups. Specifically, in the pro-drugs, at least one the R₄ groups is CH₂C(O)N(R₅)₂, CH₂C(O)OR₅, CH₂OC(O)R₅, CH(R₅)OC(O)R₅ (R, S, or RS stereochemistry), CH(R₅)C(O)R₅ (R, S or RS stereochemistry), CH₂C(R₅)₂CH₂OH, or CH₂OR₅. In particular embodiments, R₅ may be C₁-C₂₀ alkyl, aryl or aryl-alkyl which is unsubstituted or is substituted by hydroxy, oxygen, nitrogen or halogen. In particular embodiments, the pro-drugs contain identical R₄ groups.

Upon uptake by the cells, these compounds of the invention—in particular the compounds of any one of Formulae (I)-(V) can be phosphorylated such that the either of the R₄ groups is phosphate or diphosphate, while the other is hydrogen. Therefore, in particular embodiments, the metabolites of the compounds of the invention are characterized by modified R₄ groups. Therefore, in particular embodiments, in the metabolites of the compounds of the invention, at least one of the R₄ groups is phosphate or diphosphate. Preferably, in the metabolites of the compounds of the invention, one of the R₄ groups is phosphate or diphosphate, whereas the other is hydrogen.

The term “pharmaceutically acceptable salts” or “veterinary acceptable salts” as used herein means the therapeutically active non-toxic addition salt forms which the compounds of formula are able to form and which may conveniently be obtained by treating the base form of such compounds with an appropriate base or acid. The pharmaceutically acceptable acid and base addition salts as mentioned hereinabove or hereinafter are meant to comprise the therapeutically active non-toxic acid and base addition salt forms which the compounds of the invention are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulphuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form. The compounds of the invention containing an acidic proton may also be converted into their non-toxic metal or amine addition salt forms by treatment with appropriate organic and inorganic bases.

Appropriate base salt forms comprise, for example, the ammonium salts, the alkali and earth alkaline metal salts, e.g. the lithium, sodium, potassium, magnesium, calcium salts and the like, salts with organic bases. e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

Moreover, salts of acids or bases which are not physiologically acceptable may also find use, for example, in the preparation or purification of a physiologically acceptable compound. All salts, whether or not derived from a physiologically acceptable acid or base, are within the scope of the present invention.

In a preferred embodiment, the compounds of the present invention are provided as the fumarate salt.

The pro-drug salt tenofovir disproxil fumarate (TDF) may be referred to informally by those skilled in the art as simply “tenofovir”. Thus, in the context of the present invention, the term “tenofovir” may be used to mean one of more of: “tenofovir” “tenofovir disoproxil”, “tenofovir disoproxil fumarate”, “TDF”, “tenofovir alafenamide”, “tenofovir alafenamide fumarate” and “TAF”.

Equally, the term “adefovir” may be used to mean one or more of: “adefovir”, “adefovir dipivoxil” and “AD”.

### Equine Virus

### EIAV

In addition to horses (Equus caballus) EIAV can infect donkeys (Equus asinus) (Cook et al., 2001) and mules (Spyrou et al., 2003). However, a wide range of host susceptibility to disease expression is exhibited among these species (Cook et al., 2001; Hammond et al., 2000; Spyrou et al., 2003). EIAV infected horses can present three different disease states during infection: acute/sub-acute, chronic and inapparent.

The virus (EIAV) is endemic in the Americas, parts of Europe, the Middle and Far East, Russia, and South Africa. EIAV can be transmitted through blood, saliva, milk, and body secretions. Transmission is primarily through bloodsucking insects and biting flies, such as the horse-fly and deer-fly. The virus can survive up to 4 hours in the carrier. Contaminated surgical equipment and recycled needles and syringes, and bits can transmit EIAV. Further, mares can transmit EIAV to their foals via the placenta. The risk of transmitting the disease is greatest when an infected horse is ill, as the blood levels of the virus are then high.

The EIA incubation period lasts usually one to three weeks, but may be as long as three months. The most notable of the signs of disease are the concurrent development of febrile episodes (defined as rectal temperatures above 39° C.), thrombocytopenia (defined as platelet levels below 105000/μl of blood), that are typically accompanied by viremia at least of 10⁵ copies of EIAV viral particles/mL plasma.

The acute form of EIA is a sudden onset of the disease at full-force. Clinical signs include high fever, anemia (due to the breakdown of red blood cells), thrombocytopenia, weakness, swelling of the lower abdomen and legs, weak pulse, irregular heartbeat, tachypneia, petechiae on the mucous membrane, diarrhoea and blood stained feces. Thrombocytopenia is a consistent hematological finding and one of the earliest hematological abnormalities detected in acutely infected horses (Clabough et al., 1991; Crawford et al., 1996). Neurological signs are also reported in EIA infected horses (Oaks et al., 2004). Occasionally, death occurs during the acute infection, and the equine may die suddenly. After the initial bout, the majority of the horses may become asymptomatic.

The subacute form of EIA is a slower, less severe progression of the disease. Symptoms include recurrent fever, weight loss, an enlarged spleen (felt during a rectal examination), anemia, and swelling of the lower chest, abdominal wall, penile sheath, scrotum, and legs.

Some develop chronic recurring EIA signs that vary from mild illness and failure to thrive to fever, depression, petechial hemorrhages on the mucous membranes, weight loss, edema, and sometimes death. The chronic form of EIA is where an equine tires easily and is unsuitable for work. The equine may have a recurrent fever and anemia; the equine may relapse to the subacute or acute form even several years after the original attack. The majority of infected horses become life-long inapparent carriers with no overt clinical abnormalities as a result of infection (Coggins, 1984; Leroux et al., 2004; McGuire et al., 1990), yet still test positive for EIA antibodies. Such an equine can still pass on the virus.

In contrast to the pathogenesis observed in infected horses, no evident clinical signs result from EIAV in in vivo experimental infections of donkeys and mules. Indeed these Equids behave as inapparent carriers from the onset of infection (Cook et al., 2001; Spyrou et al., 2003).

EIA may cause abortion in pregnant mares. This may occur at any time during the pregnancy if there is a relapse when the virus enters the blood. Most infected mares will abort, however some give birth to healthy foals. The foals are not necessarily infected.

The present inventors have unexpectedly found that anti-viral compounds described herein are able to inhibit EIAV, and thus may be used to treat equine infectious anaemia and/or an infection with EIAV in an animal.

The in vitro data and results described herein using EIAV may be translated into an in vivo setting.

Compounds of the invention, in particular adefovir, may be used at a concentration from 1 to 10,000 nM, preferably 1 to 1000 nM, even more preferably 1 to 100 nM, to inhibit EIAV and/or in the therapy of equine infectious anaemia.

Accordingly, compounds of the invention, in particular adefovir, may be used at a dosage from 1 ng/kg to 50 mg/kg, preferably 0.05 to 25 mg/kg, even more preferably 0.1 to 10 mg/kg, to inhibit EIAV and/or in the therapy of equine infectious anaemia in a horse.

In one embodiment, the above dosages are provided every 24-120 hours, for example every 48-96 hours, for example every 72 hours, for a period of 1 to 8 weeks.

### NEV

The present inventors have identified a virus, which was obtained from horses with discordant results for EIAV testing i.e. the horses were positive for EIAV in an immunoblot but negative for EIAV in both the AGID test and an ELISA.

This previously uncharacterised virus was named New Equine Virus (NEV), as described in PCT/PT2014/000077.

The NEV virus described herein has been deposited by Equigerminal SA, Biocant Park, nucleo 4 lote 4, Cantanhede, 3060-197 Portugal under the Budapest Treaty on the International Recognition of the Deposit of Microorganisms for the purposes of Patent Procedure at European Collection of Cell Cultures (ECAAC), Culture Collections, Public Health England, Porton Down, Salisbury, Wiltshire UK SP4 0JG on 2 Dec. 2014 under accession number 14120201.

Other aspects of the present invention relate to the above viral deposit made at the ECAAC depository under accession number 14120201.

Other workers in the field may refer to NEV as EIAV or EIAV-like.

The present inventors have unexpectedly found that anti-viral compounds described herein are able to inhibit NEV, and thus may be used to treat a NEV infection and/or an infection with NEV in an animal.

The in vitro data and results described herein using NEV may be translated into an in vivo setting.

Compounds of the invention, in particular adefovir, may be used at a concentration from 0.05 nM to 28 μM, preferably 5 to 2560 nM to inhibit NEV. In particular, compounds of the invention, e.g. adefovir, may be used at a concentration above 100 nM, preferably above 1 μM to inhibit NEV.

Accordingly, compounds of the invention, in particular adefovir, may be used at a dosage from 5 ng/kg to 50 mg/kg, preferably 0.025 to 25 mg/kg to inhibit NEV. In particular, compounds of the invention, e.g. adefovir, may be used at a concentration above 0.01 mg/kg, preferably above 0.1 mg/kg to inhibit NEV in a horse.

In one embodiment, the above dosages are provided every 24-120 hours, for example every 48-96 hours, for example every 72 hours, for a period of 1 to 8 weeks.

### Equine Herpes Virus (EHV)

Several species of equine herpes virus have been described, including EHV-1, EHV-2, EHV-3, EHV-4 and EHV-5.

Equine herpesvirus 1 (EHV-1) causes abortion, respiratory disease and occasionally neonatal mortality in horses. Encephalitis can also occur in affected animals, leading to ataxia, paralysis, and death. There is a vaccine available (ATCvet code: QI05AA11), however its efficacy is questionable. Most horses have been infected with EHV-1 but the virus can become latent and show no signs or symptoms.

EHV-1 has two main strains that have been isolated. The D752 strain is more correlated to the neurological outbreak of this virus and the non-neurological outbreaks are more closely associated with N752.

Symptoms of EHV-1 infection are decreased coordination, urine dribbling, fever, hind limb weakness, leaning against things to maintain balance, lethargy and the inability to get off the ground. More signs of the infection of this virus include depression, anorexia, nasal and ocular discharges. Fever is the most common clinical sign of EHV-1.

Treatment for EHV-1 is limited, and includes the use of anti-inflammatory drugs. Vaccines exist to control the virus but not to prevent it. Treatment of EHV-1 is a particularly preferred aspect of the present invention.

In some embodiments, the EHV-1 is EHV-1 (dl TK) or EHV-1 (subtype 1) RQ dl TK, for example the EHV-1 deposited at ATCC under accession number VR-2248.

Equine herpesvirus 4 (EHV-4) causes rhinopneumonitis in horses, and is the most important viral cause of respiratory infection in foals. EHV-4 causes a lifelong latent infection in affected animals. Symptoms include fever, loss of appetite, and discharge from the nose. EHV-4 is an upper respiratory disease restricted to the infection of the respiratory tract, epithelium and its associated lymph nodes.

Equine herpesvirus 2 (EHV-2) has an uncertain role in respiratory disease in horses, but EHV-2 has been isolated from cases exhibiting symptoms such as coughing, conjunctivitis, and swollen submaxillary and parotid lymph nodes. Equine herpesvirus 3 (EHV-3) causes a disease known as equine coital exanthema. The disease is spread through direct and sexual contact and possibly through flies carrying infected vaginal discharge. Signs of the disease include pustules and ulcerations of the vagina, penis, prepuce, and perineum.

The present inventors have unexpectedly found that anti-viral compounds described herein are able to inhibit equine herpes virus, and thus may be used to treat an equine herpes virus infection and/or an infection with equine herpes virus in an animal.

The in vitro data and results described herein using EHV may be translated into an in vivo setting.

The present inventors have found that adefovir may be used at nanomolar concentrations to inhibit EHV and/or in the therapy of EHV infection. Thus, adefovir may be used at a concentration from 5 to 2500 nM, preferably 5 to 1000 nM, preferably 5 to 500 nM, preferably 5 to 150 nM, preferably 5 to 100 nM, even more preferably 5 to 10 nM.

Accordingly, adefovir may be used at a dose from 0.025 to 25 mg/kg, preferably 0.025 to 10 mg/kg, preferably 0.025 to 5 mg/kg, preferably 0.025 to 1 mg/kg, preferably 0.025 to 0.5 mg/kg, preferably 0.025 to 0.1 mg/kg even more preferably 0.025 to 0.05 mg/kg, to inhibit EHV and/or in the therapy of EHV infection in a horse.

Tenofovir may be used at a concentration from 1 to 60 μM, preferably 1 to 20 μM, preferably 1 to 10 μM, preferably 1 to 5 μM, even more preferably 1 to 3 μM, to inhibit EHV and/or in the therapy of EHV infection.

Accordingly, tenofovir may be used at a dose from 0.01 to 600 mg/kg, preferably 0.1 to 40 mg/kg, preferably 0.5 to 20 mg/kg, preferably 1 to 10 mg/kg, even more preferably 1 to 5 mg/kg, to inhibit EHV and/or in the therapy of EHV infection in a horse.

In one embodiment, the above dosages are provided every 24-120 hours, for example every 48-96 hours, for example every 72 hours, for a period of 1 to 8 weeks.

### Equine Viral Infection

The equine virus according to the invention is capable of infecting or residing in an equine. However, an equine virus may also be capable of infecting or residing in an animal other than an equine.

Examples of equine viruses include EIAV, NEV, EHV1-5, Bunyavirus, Equine Rhinovirus, Eastern Equine Encephalitis, Equine Rotavirus, Equine Adenovirus, Rabies Virus, Western Equine Encephalitis Virus, African Horse Sickness Virus (AHSV), Equine Influenza Virus, Venezuelan Encephalitis Virus, Equine Papilloma Virus, Vesicular Stomatitis Virus.

In multiple embodiments, one or more equine viruses or viral particles—preferably a detectable number of equines viruses or viral particles—has entered a host, such as a host animal. The virus may be capable of entering host cells and tissues. The virus may be dormant or replicating inside said cells and/or tissues.

Examples of equine viral diseases and infections include: African Horse Sickness, Western Equine Encephalomyelitis, Dourine, Covering Sickness, Eastem Equine Encephalomyelitis, Equine Infectious Anemia, NEV infection, Equine herpes, Equine Rhinopneumonitis, Equine Influenza, Surra, Equine Piroplasmosis, Glanders, Contagious Equine Metritis and Equine Viral Arteritis.

An infection by an equine virus according to the present invention may or may not present symptoms in the infected host.

According to the present invention, the equine viral infection may be an equine lentiviral infection. The equine virus may be an equine lentivirus.

According to the present invention, the equine lentiviral infection may be equine infectious anaemia. The equine lentivirus may be equine infectious anaemia virus (EIAV).

According to the present invention, the equine viral infection may be an equine herpesviral infection. The equine virus may be an equine herpes virus. The equine herpesvirus may be selected from the group consisting of: EHV-1, EHV-2, EHV-3, EHV-4 and EHV-5.

According to the present invention, the equine viral infection may be a New Equine Viral infection. The equine virus may be New Equine Virus (NEV).

According to the present invention, the equine viral infection may be an equine retroviral infection. The equine virus may be an equine retrovirus.

### Method of Therapy

The term “therapy” is intended to encompass any form of treatment, prevention or diagnosis, and includes treatments to both cure and prevent disease. Thus, treatment of a healthy animal is to be considered as therapy. Therapy also covers the alleviation of symptoms, in addition to curative treatments for a disease.

In one aspect, the present invention provides a method of therapy of an equine viral infection and/or infection by an equine virus in an animal comprising administering to said animal a composition comprising at least one anti-viral compound.

All embodiments described herein apply equally to a method of therapy according to the present invention.

The present invention provides a composition comprising a compound as described herein—an in particular the compounds of any one of the Formulae (I) to (V) or a pro-drug, equivalent or derivative thereof—for use as an anti-equine viral medicament.

Also provided is the use of a composition comprising at least one anti-viral compound for the manufacture of a medicament for the therapy of an equine viral infection and/or infection by an equine virus in an animal.

All embodiments described herein apply equally to such uses according to the present invention.

The therapy according to the present invention may comprise alleviating one or more clinical symptoms of an equine viral infection.

In one embodiment the animal of the invention is an equine. In another embodiment, the animal is an equine mammal. In one embodiment the animal is an equid.

Examples of equids include horses, donkeys, mules, hinnys and zebras.

In some embodiments, the terms “equid” and “equine” are used interchangeably.

In another embodiment, the animal is a horse.

Equine viruses such as EIAV, NEV and EHV can infect animals such as equines.

In another embodiment, the animal is an NEV-seropositive and/or EIAV-seropositive horse. The animal according to the present invention is not limited to an equine mammal. For example, the animal may be human.

In particular embodiments, the compounds are envisaged for use in a method of therapy comprising the reduction of viral load in an animal, such as an equine. In further particular embodiments, the compounds are envisaged for use in the reduction of clinical symptoms of the infection.

Specifically, in particular embodiments, the compounds of the invention are envisaged for use in the treatment of clinical signs or symptoms selected from the group consisting of: fever, thrombocytopenia, poor appetite, loss of body weight, wasting, anorexia, depression, malaise, apathy, lethargy, listlessness, weakness, weak pulse, irregular heartbeat, concurrent infections, low platelet count, anemia, edema, petechiation, hemorrhage, tachypneia, epistaxis (nosebleed), diarrhoea, blood-stained faeces, enlarged spleen, and swelling of the legs, abdomen, chest and/or genitals. In a further embodiment, such symptoms may be present in an equine infected with NEV and/or EIAV and/or EHV.

### Method of Diagnosis and Diagnostic Kit

The present invention provides a diagnostic method comprising:
(a) obtaining a sample from an animal;
(b) admixing with said sample a composition comprising at least one anti-viral compound;
(c) determining the presence or absence of an equine virus and/or equine viral particles and/or equine viral peptides and/or equine viral nucleic acids in said sample.

The present invention also provides a method of screening for an equine virus, said method comprising:
(a) obtaining a sample from an animal;
(b) admixing with said sample a composition comprising an anti-viral compound;
(c) determining the presence or absence of an equine virus and/or equine viral particles and/or equine viral peptides and/or equine viral nucleic acids in said sample.

The present invention also provides a method of screening for an equine virus, said method comprising:
(a) obtaining a sample from an animal;
(b) admixing with said sample a composition comprising an anti-viral compound
(c) determining the presence or absence of equine virus and/or equine viral proteins in said sample by assessing the viral reverse transcription activity in said sample, optionally wherein said viral reverse transcription activity is determined by direct contact of said sample with synthetic RNA, unlabelled and/or labelled probes.

The present invention also provides a method of screening for an equine virus, said method comprising:
(a) obtaining a sample from an animal;
(b) admixing with said sample a composition comprising an anti-viral compound
(c) determining the presence or absence of equine virus and/or equine viral proteins in said sample by assessing the viral protease activity in said sample, optionally wherein said viral protease activity is determined by direct contact of said sample with unlabelled and/or labelled probes containing cleavage sites for viral protease.

The present invention also provides a method of screening for an equine virus, said method comprising:
(a) obtaining a sample from an animal;
(b) admixing with said sample a composition comprising an anti-viral compound
(c) determining the presence or absence of equine virus and/or equine viral proteins in said sample by assessing viral replication, optionally wherein said viral replication is determined after contact of said sample with permissive cells.

In one embodiment, the equine virus may be resistant to an antiviral medicament. The equine virus may be resistant to an anti-viral compound, for example an anti-retroviral compound. The equine virus may also be resistant to a compound described herein, in particular a compound of any one of the Formulae (I) to (V) or a pro-drug, equivalent or derivative thereof.

The present invention also provides a method for identifying a viral infection in an equine mammal using any of the methods or diagnostic methods described herein.

The present invention also provides method for controlling a viral infection in a group of animals comprising the identification of a viral infection in an animal using any of the methods or diagnostic methods described herein and, optionally, the isolation of an equine virus-infected animal from other animals.

The present invention provides a kit comprising at least one anti-viral compound for use according to the invention, and optionally instructions for administration to said animal.

In one embodiment, the kit is a diagnostic kit. Such kits may be useful in the diagnosis of an equine viral infection in an animal.

By using a kit according to the present invention or by using a diagnostic method of the present invention, animals which are infected with an equine virus, such as EIAV and/or NEV and/or EHV can be identified.

Animals with an equine viral infection may be isolated from other animals (e.g. animals which do not have the equine virus). Advantageously, this helps to prevent the spread of equine viral infection from infected animals to those which are not infected, thereby controlling equine viral infection within a group of animals.

Animals with an equine viral infection may be monitored (by using a kit according to the present invention or by using a diagnostic method of the present invention) to determine the progression of the equine viral infection and/or determine the progression of equine viral disease. Animals with an equine viral infection may be isolated from other animals (e.g. animals which do not have the equine virus) once the level of infection and/or the progression of equine virus has reached a critical point. Typically animals should be isolated during febrile episodes when rectal temperatures are above 39° C., platelets levels are below 105000/μl of blood and viremia is at least of 10⁵ copies of equine viral particles/mL plasma.

In addition or alternatively, by identifying animals with an equine viral infection (by using a kit according to the present invention or by using a diagnostic method of the present invention) care should be taken to ensure that medical equipment used on an equine virus infected animal is not used on an animal which does not have a equine viral infection. Advantageously, this helps to prevent the spread of equine viral infection from infected animals to those which are not infected thereby controlling equine viral disease with a group of animals.

In some embodiments, an animal identified as having an equine virus is euthanized. Typically animals which are euthanized are those with frequent febrile episodes and animals which are lethargic or in lateral recumbence. An animal having an equine viral infection may be euthanized when the viremia peaks are frequent.

In addition to the compounds of the invention, the kits and diagnostic methods of the present invention may include but are not limited to the following techniques; competitive and non competitive assays, radioimmunoassay, bioluminescence and chemiluminescence assays, fluorometric assays, infrared assays, sandwich assays, immunoradiometric assays, dot blots, enzyme linked assays including ELISA, microtiter plates, antibody coated strips, or dipsticks for rapid monitoring of urine or blood, and immunocytochemistry, DNA or RNA amplification techniques including polymerase chain reaction, reverse transcription and LAMP assays. For each kit the range, sensitivity, precision, reliability, specificity and reproducibility of the assay are established. Intraassay and interassay variation is established at 20%, 50% and 80% points on the standard curves of displacement or activity.

The sample as referred to herein is obtained/obtainable from an animal. In one embodiment, the sample is obtained/obtainable from an equine such as a horse, a donkey, a mule, a hinny, or a zebra.

The sample may be blood, blood serum, plasma, saliva, sputum, urine, fecal biopsy, lymph node biopsy, milk, semen, and/or sweat.

The diagnostic methods of the present invention are typically carried out ex vivo or in vitro.

Compositions according to the invention and/or a pharmaceutical composition according to the invention may also be used in a kit or assay for the diagnosis or prevention of an equine viral disease, and/or the diagnosis or prevention of an infection by an equine virus in an animal.

### Dosage and Administration

The present invention provides a pharmaceutical composition comprising at least one anti-viral compound (for example an anti-retroviral compound) for use according the invention and a pharmaceutically acceptable carrier, vehicle, diluent or excipient.

### Administration

The compounds of the present invention may be formulated for oral or parenteral use in a conventional manner using known pharmaceutical carriers and excipients, and they may be presented in unit dosage form or in multiple dose containers. The compositions may be in the form of tablets, capsules, solutions, suspensions or emulsions. These compounds may also be formulated as suppositories utilizing conventional suppository bases such as cocoa butter or other fatty materials. The compounds may, if desired, be administered in combination with other antiviral compounds.

In one embodiment, the composition for use according to the invention may be administered via a route selected from the group consisting of: oral, parenteral, intravenous, intramuscular, subcutaneous, intranasal, intrapulmonary, intraperitoneal, intradermal, intrathecal and epidural.

In a preferred embodiment, the route of administration is oral, intravenous or intramuscular. In a particularly preferred embodiment, the route of administration is intramuscular, for example injectable intramuscular.

The present invention further provides formulations of the compounds of the present invention, which are particularly suited for the therapeutic use envisaged. The compounds of the invention may be formulated with conventional carriers and excipients, which will be selected in accordance with ordinary practice. Tablets may contain excipients, glidants, fillers, binders and the like. Aqueous formulations may be prepared in sterile form, and when intended for delivery by other than oral administration generally will be isotonic. Formulations optionally contain excipients such as those set forth in the “Handbook of Pharmaceutical Excipients” (1986) and include sodium hydroxide, ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” or “veterinary acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier or veterinary acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol, benzyl alcohol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-step procedure, with the selected carrier material and, where appropriate, the other additives such as surface-active agents may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgents or emulsifiers, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable from coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalenesulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphosphatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with polypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having hydrocarbon radicals optionally substituted with halo, phenyl, substituted phenyl or hydroxy; for instance quaternary ammonium salts containing as N-substituent at least one C₈-C₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2nd Ed. (Hanser Verlag, Vienna, 1981) and “Encyclopaedia of Surfactants”, (Chemical Publishing Co., New York, 1981).

While it is possible for the active ingredients to be administered alone it is preferable to present them as pharmaceutical formulations. The formulations for pharmaceutical or veterinary use of the present invention comprise at least one active ingredient, as above described, together with one or more pharmaceutical or veterinary acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral or parenteral (including subcutaneous, intraperitoneal, intramuscular, intravenous, intradermal, intrathecal and epidural) administration.

The formulations may conveniently be presented in unit dosage form and may be prepared by any of the methods well known in the art of pharmacy. In particular embodiments, as indicated above, the compounds of the present invention are provided as oral or injectable formulations.

Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets, enteric capsules or tablets each containing a predetermined amount of the active ingredient as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

The formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane-1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Preferred unit dosage formulations are those containing an effective dose, as hereinabove recited, or an appropriate fraction thereof, of an active ingredient. It should be understood that in addition to the ingredients particularly mentioned above the formulations of this invention may include other agents conventional in the pharmaceutical or veterinary art having regard to the type of formulation in question, for example those suitable for oral administration may include flavouring agents.

Compounds of the invention can be provided as controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations are adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinylpyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and the like. Depending on the route of administration, the veterinary composition may require protective coatings. Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several compounds or active ingredients are used in combination, they do not necessarily bring out their joint therapeutic effect directly at the same time in the animal to be treated, the corresponding composition may also be in the form of a medical kit or package containing the two or more ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection.

### Dosage

The optimal dosage regimen for the treatment of an animal infected with equine virus may be achieved when the compound according to the invention is administered at least once weekly, with a total dose of 10 to 1000 mg/kg. Such a regimen ensures reduction of the viral load and/or reduction of clinical symptoms in an animal infected with an equine virus. Thus, a further aspect of the present invention provides the compounds of the present invention, for use in the treatment methods of the present invention, wherein the compound is administered at least once weekly, with a total dose of 10 to 1000 mg/kg. In certain embodiments, the compound is administered via oral route. In certain embodiments, the compound is administered via subcutaneous injections.

In one embodiment, the at least one compound of the present invention is provided at a total dose of 10-1000 mg/kg, during 1 to 6 weeks.

In another embodiment, a compound of the invention, in particular adefovir, may be administered to a horse infected with an equine virus (e.g. NEV, EIAV or EHV) at a dosage of from 0.1 to 5 mg/kg every 24-120 hours, for example every 48-96 hours, for example every 72 hours, for a period of 1 to 8 weeks.

In addition, when provided in unit dosage forms, the compositions may contain from about 0.1 to about 100 mg/kg/dose of the active anti-viral ingredient. The dosage of the compounds of the invention is dependent on such factors as the weight and age of the animal, as well as the particular nature and severity of the disease, and within the discretion of the physician or veterinary practitioner. The dosage for treatment may vary depending on the frequency and route of administration.

A dosage can be divided into one, two or more doses in a suitable form to be administered at one, two or more times throughout a given time period.

The compositions of the invention can be administered for prophylactic or therapeutic treatments. In prophylactic applications, compositions can be administered to an animal with a clinically determined predisposition or increased susceptibility to development of an equine viral infection or disease. Compositions of the invention can be administered to the animal in an amount sufficient to delay, reduce, or preferably prevent the onset of clinical disease or infection. In therapeutic applications, compositions are administered to an animal already suffering from disease or infection in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or infection. A therapeutically effective amount of a compound may not be required to cure a disease or infection but will provide a treatment for a disease or infection such that the onset of the disease or condition is delayed, hindered, or prevented, or the disease or infection symptoms are ameliorated, or the term of the disease or infection is changed or, for example, is less severe or recovery is accelerated in an animal. Amounts effective for this use may depend on the severity of the disease or infection and the weight and general state of the animal, but generally range from about 0.5 mg to about 3000 mg of the agent or agents per dose per animal. Suitable regimes for initial administration and booster administrations may be typified by an initial administration followed by repeated doses at one or more hourly, daily, weekly, or monthly intervals by a subsequent administration. The total effective amount of a compound or compounds present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more compounds present within the compositions of the invention and used in the methods of this invention applied to animals (e.g., humans or equines) can be determined by the ordinarily-skilled artisan with consideration of individual differences in age, weight, and the condition of the animal. The compositions of the invention are administered to an animal in an effective amount, which is an amount that produces a desirable result in a treated animal (e.g. the slowing or remission of infection). Therapeutically effective amounts can be determined empirically by those of skill in the art.

The animal may also receive an agent in the range of about 0.1 to 3,000 mg per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) mg dose per week. An animal may also receive an agent of the composition in the range of 0.1 to 3,000 mg per dose once every two or three weeks.

Single or multiple administrations of the compositions of the invention comprising an effective amount can be carried out with dose levels and pattern being selected by the treating physician or veterinarian. The dose and administration schedule can be determined and adjusted based on the severity of the disease or infection in the animal, which may be monitored throughout the course of treatment according to the methods commonly practiced by clinicians, veterinarians or those described herein.

The compositions of the present invention may be used in combination with either conventional methods of treatment or therapy or may be used separately from conventional methods of treatment or therapy.

When the compositions of this invention are administered in combination therapies with other agents, they may be administered sequentially or concurrently to an animal. Alternatively, pharmaceutical compositions according to the present invention may be comprised of a combination of a composition of the present invention in association with a pharmaceutically acceptable excipient, as described herein, and another therapeutic or prophylactic agent known in the art.

Additional antiviral compounds which may be used in conjunction with the present invention include, but are not limited to: zidovudine (AZT), darunavir, daclatasvir and indinavir, or a pro-drug, equivalent or derivative thereof.

### Other Uses

Also provided is the use of a composition comprising at least one anti-viral compound for modulating reverse transcriptase activity in an equine virus.

Reverse transcriptase activity may be increased or inhibited by the compound, preferably inhibited. Reverse transcriptase activity may be determined by any suitable method in the art. Such methods are within the capability of the skilled person.

Also provided is the use of a composition comprising at least one anti-viral compound for inhibiting the replication of an equine virus in vitro.

Techniques for monitoring the replication of an equine virus are known to those skilled in the art. Examples include viral titre and enumeration assays and RT qPCR.

Also provided is the use of a composition comprising at least one anti-viral compound for promoting the survival of an animal cell infected with an equine virus in vitro.

In one embodiment, said animal cell is an equine cell, for example an equine dermal cell or equine macrophage.

Suitable equine dermal cells include those bearing the reference ATCC CCL57.

Suitable equine macrophage cells include those denoted as “MacF” or those which were spontaneously immortalized from a NEV-seropositive horse accordingly the procedures reported in Fidalgo-Carvalho et. al, 2009.

Techniques for monitoring cell survival are known to those in the art and may include cell enumeration, trypan blue staining, flow cytometry, apoptosis assays, western blotting, ELISA, immunohistochemistry, cell viability assays and formazan-based assays.

### EXAMPLES

The present invention is further described by way of the following non-limiting examples:

### Example 1: NEV Possess the Morphology of a Lentivirus

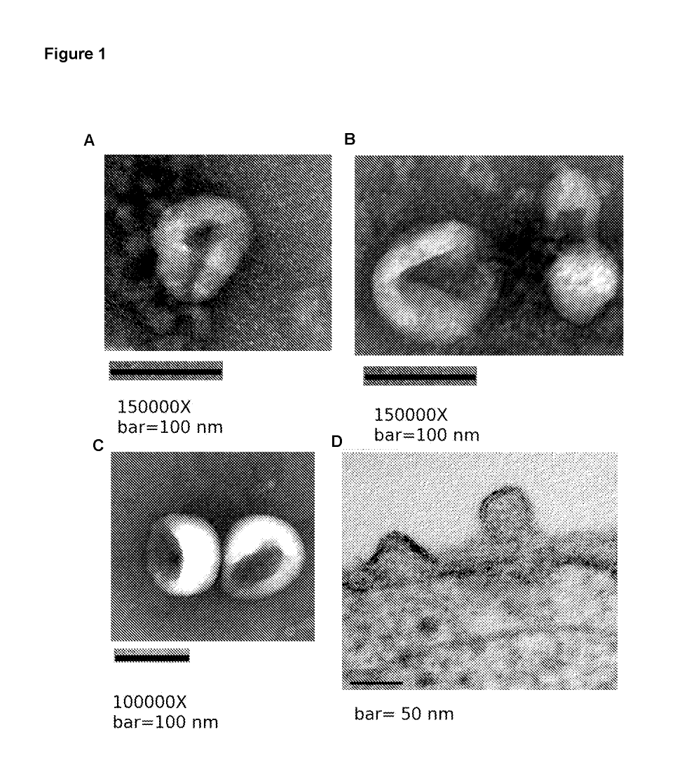

NEV viral particles were layered on the top of 20% sucrose gradient and ultracentrifuged at 50000 g for 1 hour at 4° C. Pellets were dissolved in phosphate buffer and submitted to negative staining electron microscopy. FIGS. 1A, 1B and 1C show that NEV viral particles range from 60 to 120 nm and possess a lentiviral morphology with an ellipsoid shaped core.

Moreover, NEV infected cells were also analysed by electron microscopy after 5 days of infection. FIG. 1D shows viral particles budding from plasma membranes similarly to lentiviral viral particles.

### Example 2: Adefovir Dipivoxil Possess Antiviral Activity Against Equine Lentivirus

Equine Dermal cells (ATCC CCL57) (10⁵ cells/cm²) were seeded in 96 well plates 24 to 72 hours before infection in complete media and incubated in a humid chamber at 37° C. and 5% CO₂ until 95%-99% confluent monolayers were attained. Complete media was composed of DMEM medium (Gibco, Life Technologies) with 10% Inactivated Fetal bovine serum (Gibco, Life Technologies), 1% Glutamax (Gibco, Life Technologies) and 1% Penicillin-Streptamicin (Gibco, Life Technologies).

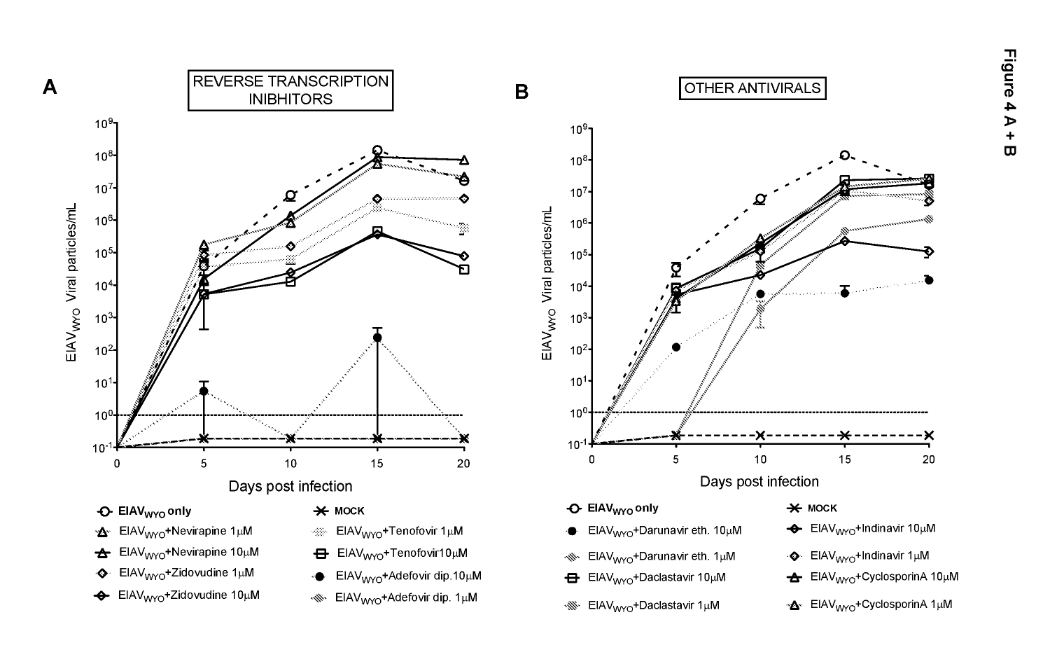

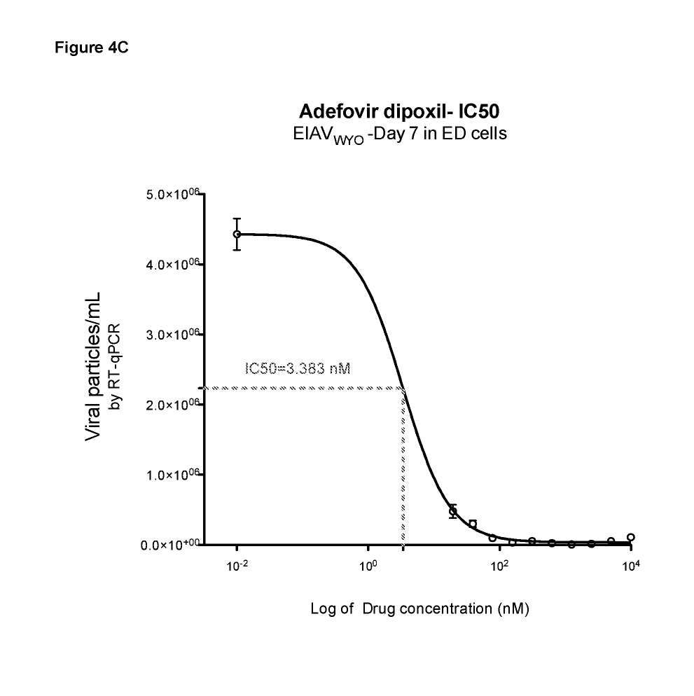

The antiviral effect of adefovir dipivoxil, as well as other are antiviral drugs approved by FDA or EMA such as Zidovudine (nucleoside reverse transcription inhibitor), Nevirapine (non-nucleoside reverse transcription inhibitor), Indinavir sulphate (a HIV-1 protease inhibitor), Darunavir Ethanolate (a HIV-1 protease inhibitor), Daclastavir (HCV NS5A inhibitor), Cyclosporin A (immunosuppressive agent) and the drug that is being evaluated in pre-clinical/clinical assays as antivirals, such as Tenofovir (a nucleoside reverse transcription inhibitor), was evaluated (FIGS. 4A and 4B). Cells were pre-treated with drugs at 1 or 10 μM in quintuplicates for one hour and infected with NEV (1130 PFU per well) for 2 hours. After 2 hours fresh media with drugs was replaced. Cells were then incubated for 9 days in a humid chamber 37° C. and 5% CO2. After 9 days cellular viability of treated cells was compared to those of non infected cells and infected and not treated cells by using PrestoBlue cell viability reagent. Results showed that cellular viability of NEV untreated cells was near zero, and that Adefovir dipivoxil completely reverted the effect of NEV on cellular viability. Adefovir dipivoxil treated cells showed cellular viability similar to non-infected cells. The differences observed between NEV infected cells treated with adefovir dipivoxil and non-treated NEV infected cells were significant statistically (p<0.0001) for both drug concentrations of 1 or 10 μM. Moreover, treatment of infected cells with tenofovir at 10 μM also increased the cell viability of infected cells. The differences observed between tenofovir-treated and non treated cells infected cells was significant statistically for p<0.005.

### Example 3: Analysis of Antiviral Activity of Adefovir Dipivoxil Against NEV

### Equine Dermal Cells

Equine Dermal cells (ATCC CCL57) (10⁵ cells/cm²) were seeded in 96 well plates 24 to 72 hours before infection in complete media and incubated in a humid chamber at 37° C. and 5% CO₂ until 95%-99% confluent monolayers were attained. Complete media was composed of DMEM medium (Gibco, Life Technologies) with 10% Inactivated Fetal bovine serum (Gibco, Life Technologies), 1% Glutamax (Gibco, Life Technologies) and 1% Penicillin-Streptamicin (Gibco, Life Technologies).

Confluent cell monolayers were washed twice with HBSS to remove non-adherent cells, incubated with 150 μl of infection media (DMEM with 5% FBS) and pre-treated with adefovir dipivoxil [from a stock of 10 mM in 100% DMSO] (Selleckchem, Germany). The pre-treatment with adefovir dipivoxil (Selleckchem, Germany) preceded the infection. To determine drug IC₅₀ ten 1:2 serial dilutions ranging from 5 to 2560 nM concentrations were performed in quintuplicates for each drug concentration. ED cells were pre-treated with drug for 60 minutes before infection, and treatment maintained during infection. NEV viral particles (10 μl containing 1130 PFU) were added to the cell culture media for 2 hours. After infection, cells were washed twice with HBSS to remove unbound virus and fresh complete media and fresh drug was replaced in each well. Eleven days after infection, cells were screened for viability by using the PrestoBlue cell viability assay (Molecular Probes, Life Technologies) accordingly to manufacturer instructions. Absorbance (570 nm) of adefovir dipivoxil treated cells was compared to those of non-infected and/or untreated NEV-infected cells. 15 μl Prestoblue reagent was added directly to the assay wells and absorbance measured at 570 nm after 24 hours of incubation with the Prestoblue reagent. All the absorbance values were corrected by removing the baseline absorbance values of the Prestoblue reagent incubated 24 hours without cells.

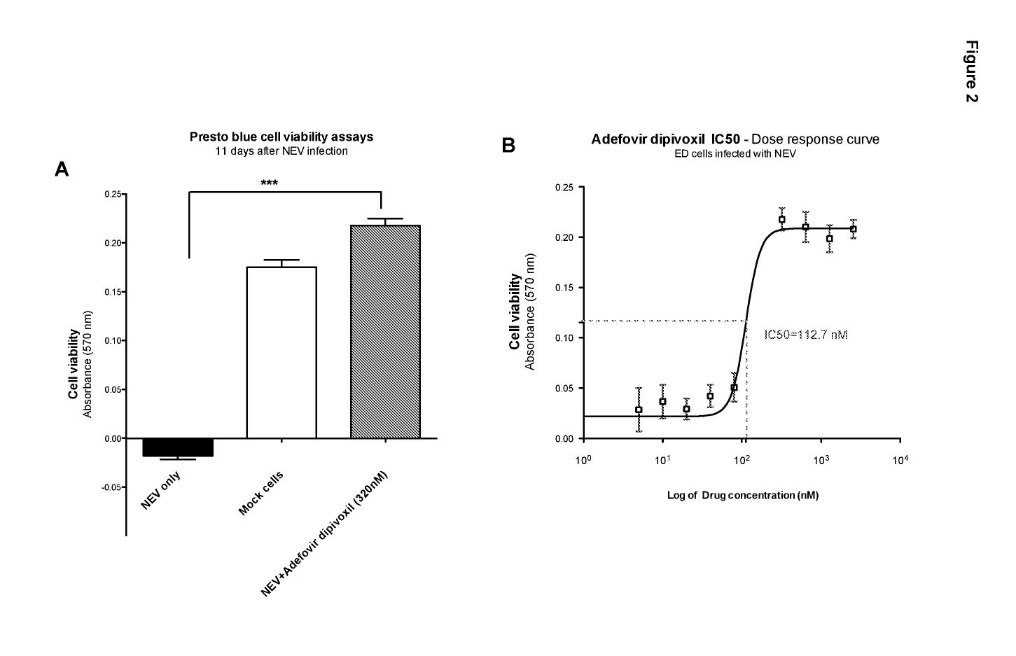

FIG. 2A demonstrates that the cell viability of ED cells at 11 days post NEV infection was very similar to zero, confirming the high NEV cytopathicity. In contrast to that, non-infected cells (mock cells) and cells treated with 320 nM of adefovir dipivoxil showed very similar absorbance values, suggesting that the drug could revert cell viability of ED infected cells. The results indicate that adefovir dipivoxil can block the NEV cytopathic effects in ED cells after a single dose treatment at nanomolar concentrations. The differences observed between NEV treated and untreated cells were statistically significant by using 2-way ANOVA and Bonferroni post tests with a p value <0.0001.

FIG. 2B shows the dose response curve of cell viability of NEV infected cells in the presence of ten serial dilutions (1:2) of adefovir dipivoxil with concentrations ranging from 5 to 2560 nM. The results were analysed by Prism software by using the four-parameter function (log (drug) vs. response assuming a variable slope). The results showed that the IC₅₀ of the drug was 112.7 nM (degrees of freedom=51, R²=0.9460, Hill slope=4.784).

### Equine Macrophage Cell Lines

An equine macrophage cell line (MacF) was spontaneously immortalized from a NEV-seropositive horse accordingly the procedures reported in Fidalgo-Carvalho et. al, 2009.

Mac F cells were seeded in complete media and incubated in a humid chamber at 37° C. and 5% CO₂ at a cell density of 0.5×10⁵ cells/cm² in 96 well plates. Cells were incubated 24 to 72 hours before NEV infection until 95%-99% confluent cell monolayers were attained.

Confluent cell monolayers were washed twice with HBSS to remove non-adherent cells, incubated with 150 μl infection media (DMEM with 5% FBS) and pre-treated with adefovir dipivoxil [from a stock of 10 mM in 100% DMSO] (Selleckchem, Germany), as reported above for ED cells. To determine drug IC₅₀ in MacF cell line, eleven serial dilutions of 1:4 ranging from 0.05 nM to 28 μM were performed in quintuplicates for each drug concentration. MacF Cells were pre-treated with drug for 60 minutes before infection, and treatment was maintained during infection. NEV viral particles (10 μl containing 1130 PFU) were added to the cell culture media for 2 hours. After infection, cells were washed twice with HBSS to remove unbound virus and fresh complete media and fresh drug was replaced in each well. Eleven days after infection, cells were screened for viability by using the PrestoBlue cell viability assay (Molecular Probes, Life Technologies) accordingly to manufacturer instructions. Absorbance (570 nm) of adefovir dipivoxil treated cells was compared to those of non-infected and/or untreated NEV infected cells. 15 μl of Prestoblue reagent was added directly to the assay wells and absorbance measured at 570 nm after 24 hours of incubation with the Prestoblue reagent. All the absorbance values were corrected by removing the baseline (absorbance values of the Prestoblue reagent incubated 24 hours without cells).

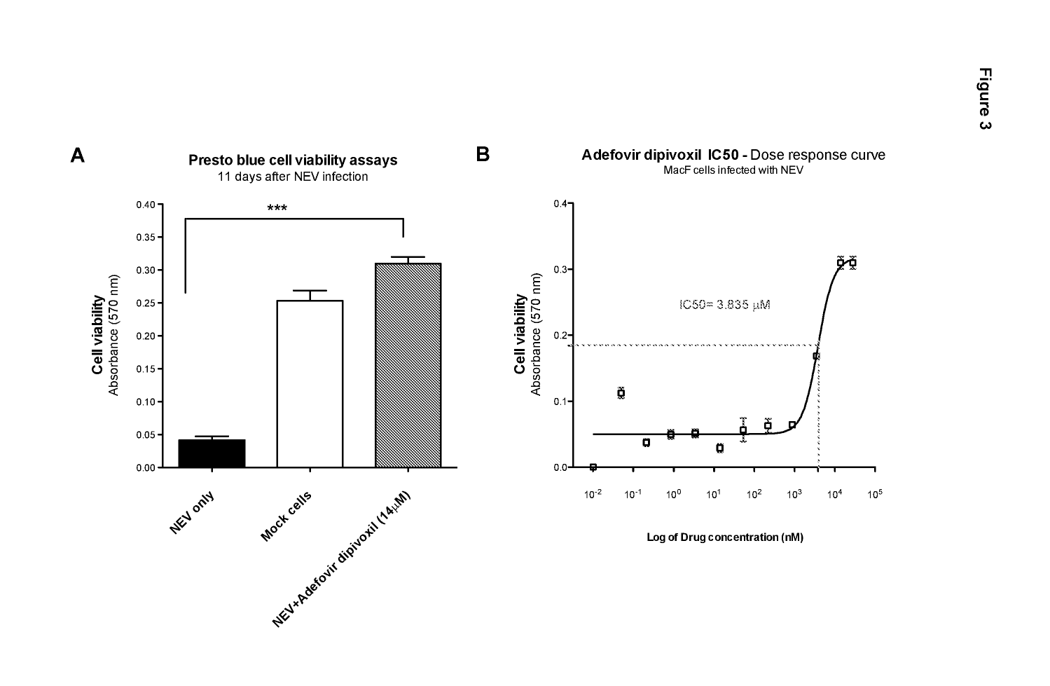

FIG. 3A demonstrates the absence of cell viability of MacF cells at 11 days post NEV infection, confirming the NEV cytopathic effects in the macrophage cell line, MacF. However, absorbance values of Mock MacF cells were very similar (or even slightly higher) to those observed for cells treated with 14 μM adefovir dipivoxil. The results indicate that adefovir dipivoxil could also block the NEV cytopathic effects in macrophage cell lines established from NEV-seropositive horses.

FIG. 3B demonstrates the dose response curve of cell viability of macrophage cell lines infected with NEV in the presence of eleven serial dilutions (1:4) of adefovir dipivoxil with concentrations ranging from 0.05 nM to 28 μM. The results were analysed by Prism software by using the four-parameter function (log (drug) vs. response assuming a variable slope). The results showed that the IC₅₀ of the drug was 3.835 μM (degrees of freedom=56, R²=0.9146, Hill slope=2.251).

The adefovir dipivoxil IC₅₀ was 3.835 μM for MacF cells and 112.7 nM for ED cells.

Being MacF cells of macrophage origin and obtained from NEV-seropositive horses could suggest the in vivo efficacy of this antiviral drug against NEV infection.

### Example 4: Adefovir Dipivoxil Possess Antiviral Activity Against EIAV

Adefovir dipivoxil is highly active against Equine Infectious Anemia Virus (EIAV).

Equine dermal cells (3×10⁴ cells per well of a 96-well plate) were seeded and incubated in complete media (as described above for NEV infections) under 5% CO₂ at 37° C., for 24 h to 72 hours prior to infection. The antiviral effect adefovir dipivoxil, as well as other are antiviral drugs approved by FDA or EMA such as Zidovudine (nucleoside reverse transcription inhibitor), Nevirapine (non-nucleoside reverse transcription inhibitor), Indinavir sulphate (a HIV-1 protease inhibitor), Darunavir Ethanolate (a HIV-1 protease inhibitor), Daclastavir (HCV NS5A inhibitor), Cyclosporin A (immunosuppressive agent) and the drug that is being evaluated in pre-clinical/clinical assays as antivirals, such as Tenofovir (a nucleoside reverse transcription inhibitor), was evaluated (FIGS. 4A and 4B).

The drugs mentioned above were also tested as antivirals for EIAV. For that cells were pre-treated with drugs at two different concentrations 1 or 10 μM for 1 hour in triplicates. Cells were then infected with EIAV_{WYO} (3 MOI for 2 hours). After infection fresh media and drugs were replaced and cells incubated for 5, 10, 15 and 20 days. The EIAV_{WYO} replication was evaluated by quantifying EIAV viral genome in culture supernatants by means of RT qPCR. FIGS. 4A and 4B show the effect of the different drugs in EIAV replication kinetics. Remarkably Adefovir dipivoxil blocked the EIAV replication in a single dose. Virus was only recovered at low levels from treated cells in one of three replicate at day 5 (10 viral particles/mL) and another of three replicates at day 15 (100 viral particles/mL). The differences observed between the treated and not treated infected cells were statistically significant for p<0.001. Moreover, tenofovir and zidovudine at 10 μM also had a significant effect in the replication of EIAV by significantly reducing the viral replication form 10⁸ to 10⁵ viral particles/mL at day 15 and 20 post infection. Also, protease inhibitors showed marked effect on EIAV viral replication. At day 15 post infection Darunavir in the concentration of 10 μM significantly decreased viral replication from 10⁸ to 10³ viral particles/mL, and Indinavir at 10 μM significantly decreased viral replication from 10⁸ to 10⁴ viral particles/mL.

Adefovir dipivoxil showed marked antiviral activity against Equine infectious anemia virus (FIGS. 4A and 4B).

Furthermore, to better evaluate the effect of the drug on EIAV_{WYO} viral replication on ED cells, we have determined the IC₅₀ of adefovir dipivoxil. Confluent cell monolayers were seeded as described above, washed twice with HBSS to remove non-adherent cells, incubated with 150 μl of infection media (DMEM with 5% FBS) and pre-treated with adefovir dipivoxil [from a stock of 10 mM in 100% DMSO] (Selleckchem, Germany) for 60 minutes. The pre-treatment with Adefovir dipivoxil (Selleckchem, Germany) preceded the infection. Eleven serial dilutions ranging from 0.01 to 10,000 nM (0.01, 19.53, 39.06, 78.130, 156.250, 312.5, 625, 1250, 2500, 5000 and 10000 nM) were performed in triplicates for each drug concentration. Treatment was maintained during infection. EIAV_{WYO} viral particles (10 μl containing 1.9×10⁵ viral particles) were added in each well of a 96-well plate and incubated for 2 hours. After infection, cells were washed twice with HBSS to remove unbound virus and fresh complete media containing fresh drug was replaced in each well. Seven days after infection, wells were screened for number of viral particles/mL of cell culture supernatant using RT qPCR techniques. For quantification, a standard curve of an EIAV plasmid DNA was used. To obtain the standard curve, seven 1:10 dilutions in duplicates were tested in parallel. Samples were tested in triplicates. The standard curve obtained (with R² 0.994, Efficiency 96.56%) allowed us to quantify the viral particles.

FIG. 4C demonstrates the effect of adefovir dipivoxil on EIAV_{WYO} viral replication in ED cells. The results were analysed by Prism software using the four-parameter function (log (drug) vs. response assuming a variable slope) and showed that the IC₅₀ of the drug was 3.383 nM (degrees of freedom=29, R²=0.9917, Hill slope=0.4492).

### Adefovir Dipivoxil CC₅₀ in Equine Dermal Cells

Furthermore we addressed cytotoxicity of adefovir dipivoxil in ED cells. For that, ED cells (10⁵ cells/cm²) were seeded as mentioned above in 96-well plates. Confluent ED cell monolayers were washed twice with HBSS to remove non-adherent cells and pre-treated with adefovir dipivoxil [from a stock of 10 mM in 100% DMSO] (Selleckchem, Germany) for 6 days. To determine drug CC₅₀, twelve different concentrations of 4, 6, 9, 13.5, 20.250, 30.370, 45.560, 68.340, 102.520, 153.770, 230.660 and 2000 μM were performed in quintuplicates per each drug concentration. Cell viability was assessed by using the PrestoBlue reagent as mention above for IC₅₀ analysis and incubated for 24 hours.

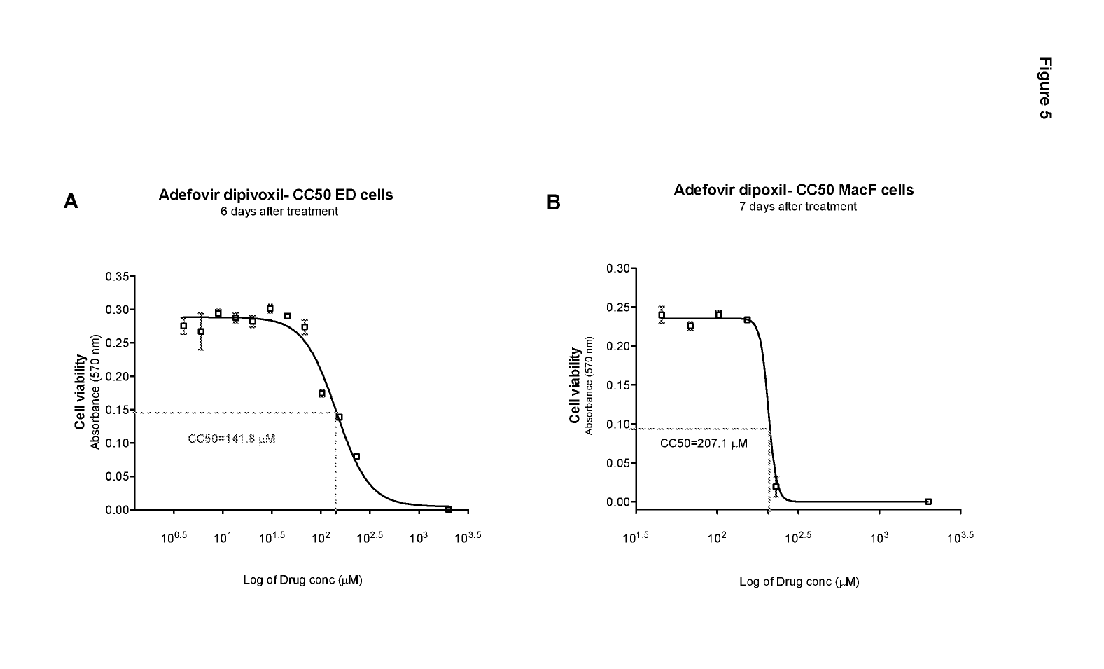

FIG. 5A shows the dose response curve of cell viability in the presence of different concentrations of adefovir dipivoxil. The results were analysed by Prism software by using the four-parameter function (log (drug) vs. response assuming a variable slope). The results showed that the CC₅₀ of the drug was 141. 80 μM (degrees of freedom=55, R²=0.9602, Hill slope=2.427).

### Adefovir Dipivoxil CC₅₀ in Macrophage Cells Lines

Cytotoxicity of adefovir dipivoxil in MacF cells (10⁵ cells/cm²) were seeded as mentioned above in 96-well plates. Confluent MacF cell monolayers were washed twice with HBSS to remove non-adherent cells and pre-treated with adefovir dipivoxil [from a stock of 10 mM in 100% DMSO] (Selleckchem, Germany) for 7 days. To determine drug CC₅₀, six different concentrations of 45.560, 68.340, 102.520, 153.770, 230.660 and 2000 μM were performed in quintuplicates per each drug concentration. Cell viability was assessed by using the PrestoBlue reagent as mention above for IC₅₀ analysis and incubated for 24 hours.

FIG. 5B shows the dose response curve of cell viability in the presence of different concentrations of adefovir dipivoxil. The results were analysed by Prism software by using the four-parameter function (log (drug) vs. response assuming a variable slope). The results showed that the CC₅₀ of the drug was 207. 10 μM (degrees of freedom=25, R²=0.9895, Hill slope=17.08).

### Example 5: Adefovir Dipivoxil Possess Antiviral Activity Against Equine Herpesvirus-1 (EHV-1)

### Antiviral Activity Assay in Equine Dermal Cells

Antiviral activity of Adefovir dipivoxil was evaluated directly by measuring the reduction in number of EHV-1 viral particles in supernatant and indirectly by quantifying the upturn of viable cells numbers in infected cultures.

Equine Dermal cells (ATCC CCL57) (10⁵ cells/cm²) were seeded in 96 well plates 24 to 72 hours before infection in complete media and incubated in a humid chamber at 37° C. and 5% CO₂ until 95%-99% confluent monolayers were attained. Complete media was composed of DMEM medium (Gibco, Thermofisher Scientific) with 10% of Inactivated Fetal bovine serum (Gibco, Thermofisher Scientific), 1% of Glutamax (Gibco, Life Technologies) and 1% Penicilin-Streptamicin (Gibco, Thermofisher Scientific). Confluent cell monolayers were washed twice with HBSS to remove non-adherent cells, incubated with 150 μl of infection media (DMEM with 5% FBS) and pre-treated with different concentrations of Adefovir dipivoxil [from a stock of 100 mM in 100% DMSO] (Selleckchem, Germany). The pre-treatment with the drug preceded the infection.

ED cells were pre-treated with drug for 30 minutes before infection, and treatment maintained during infection.

To determine the IC₅₀ of Adefovir dipivoxil eight or ten 1:2 serial dilutions ranging from 5 to 2500 nM concentrations were performed in quintuplicates per each drug concentration. ED cells were pre-treated with drug for 30 minutes before infection, and treatment maintained during infection.

Cells were infected with EHV-1 at 1 MOI for 2 hours. Infective EHV-1 viral particles were produced in ED cells with an EHV-1 molecular clone purchased from ATCC (VR-2248). After infection cells were washed twice with HBSS to remove unbound virus and fresh complete media and fresh drug was replaced in each well. Treatment was maintained during 6 days without addition of fresh drug.

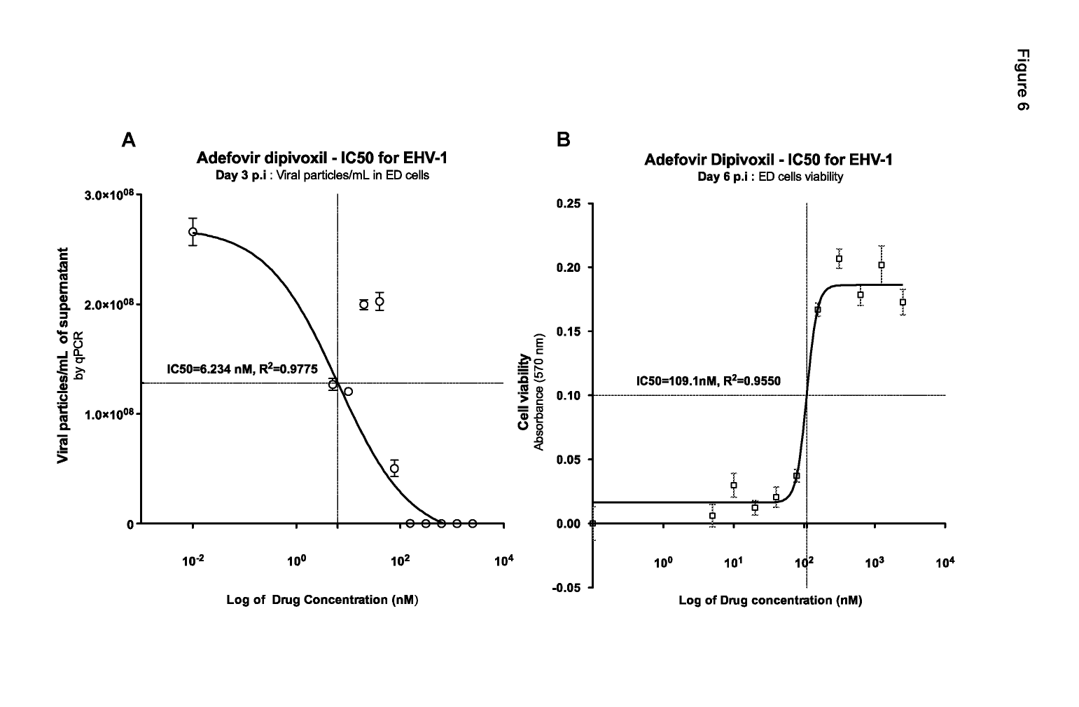

Quantification of EHV-1 viral particles was assayed 66 h or 72 h (3 days) post infection. Wells were screened for number of viral particles/mL of cell culture supernatant by using the EHV-1 specific quantitative Real Time PCR assay (CFX96 Biorad apparatus). A standard curve of an EHV-1 synthetic DNA was used. To obtain the standard curve, seven 1:10 dilutions of the synthetic DNA in duplicates were tested in parallel. Samples were tested in triplicates. The standard curve obtained (with R2 0.995, Efficiency 91.7%) allowed us to quantify the EHV-1 viral particles produced into the cell culture media. The results were analysed by Prism software by using the four-parameter function (log (drug) vs. response assuming a variable slope). The retrieved IC₅₀ in three independent experiments measured by qPCR at day 3 post infection was 4.415 nM with a standard deviation of 2.727. FIG. 6A show a representative experiment of IC₅₀ values determined by qPCR at day 3.

After 6 days post-infection reazurin-based cell viability assays (PrestoBlue, Thermofisher Scientific) were used to evaluate the increased cell numbers in the same conditions as indicated above for qPCR quantification. For cell viability assays absorbance (570 nm) of EHV-1 infected cells treated with different concentrations of Adefovir dipivoxil were compared to those of non-infected and/or not treated EHV-1 infected cells. 15 μl of Prestoblue reagent was added directly to the assay wells and absorbance measured at 570 nm after 24 hours of incubation with the Prestoblue reagent. All the absorbance values were corrected by removing the baseline, absorbance values of the Prestoblue reagent incubated 24 hours without cells. The results were analysed by Prism software by using the four-parameter function (log (drug) vs. response assuming a variable slope). FIG. 6B show a representative experiment of dose curve responses and IC₅₀ value determined at day 6 post infection. The average IC₅₀ value obtained in three independent experiments was 122.9 nM, with a standard deviation of 19.414.

Adefovir dipivoxil showed an excellent therapeutic index (CC₅/IC₅₀) of 1162.3 for EHV-1.

### Antiviral Activity Assay in Equine Macrophage Like Cell Lines

Antiviral activity of Adefovir dipivoxil was also evaluated in macrophage like cell lines. MacF cells were seeded at a density of 10⁵ cells/cm² in 96 well plates 24 to 72 hours before infection in complete media and incubated in a humid chamber at 37° C. and 5% CO₂ until 95%-99% confluent monolayers were attained. Complete media was composed of DMEM medium (Gibco, Thermofisher Scientific) with 10% of Inactivated Fetal bovine serum (Gibco, Thermofisher Scientific), 2% of Glutamax (Gibco, Thermofisher Scientific) and 1% Penicilin-Streptamicin (Gibco, Thermofisher Scientific). Confluent cell monolayers were washed twice with HBSS to remove non-adherent cells, incubated with 150 μl of infection media (DMEM with 5% FBS) and pre-treated with different concentrations of Adefovir dipivoxil [from a stock of 100 mM in 100% DMSO] (Selleckchem, Germany). The pre-treatment with the drug preceded the infection.

MacF cells were pre-treated with drug for 30 minutes before infection, and treatment maintained during infection.

To determine the IC₅₀ of Adefovir dipivoxil eight 1:2 serial dilutions ranging from 13 to 500 nM concentrations were performed in quintuplicates per each drug concentration. MacF cells were pre-treated with drug for 30 minutes before infection, and treatment maintained during infection.

Infections studies, viral particle quantification and cell viability assays were performed as described above for antiviral assays in ED cells.

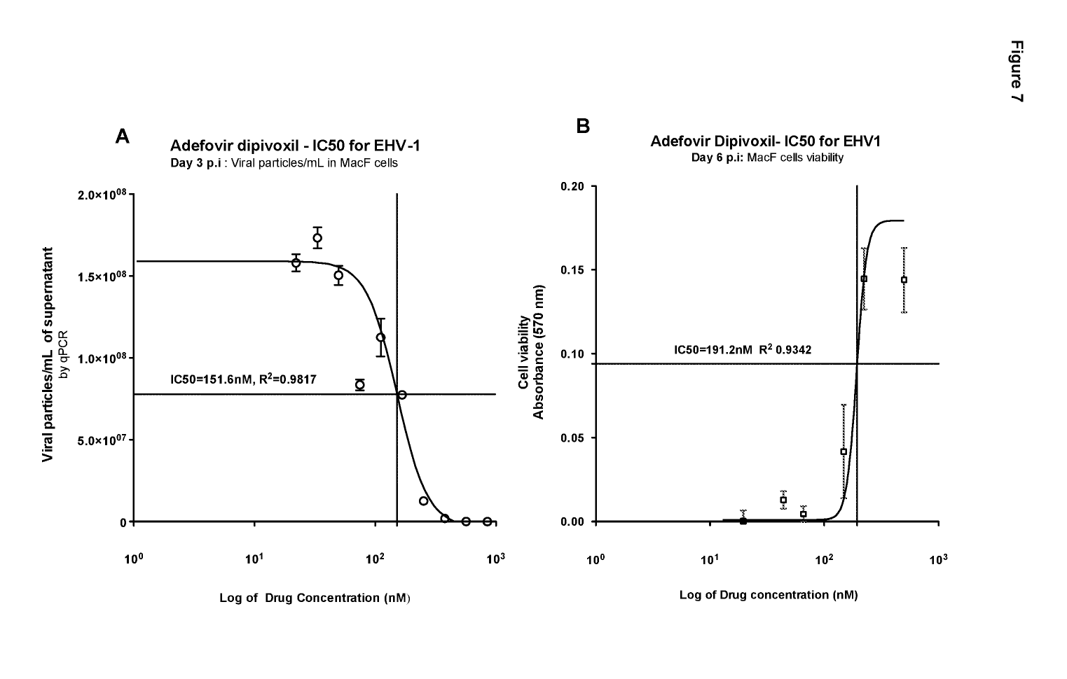

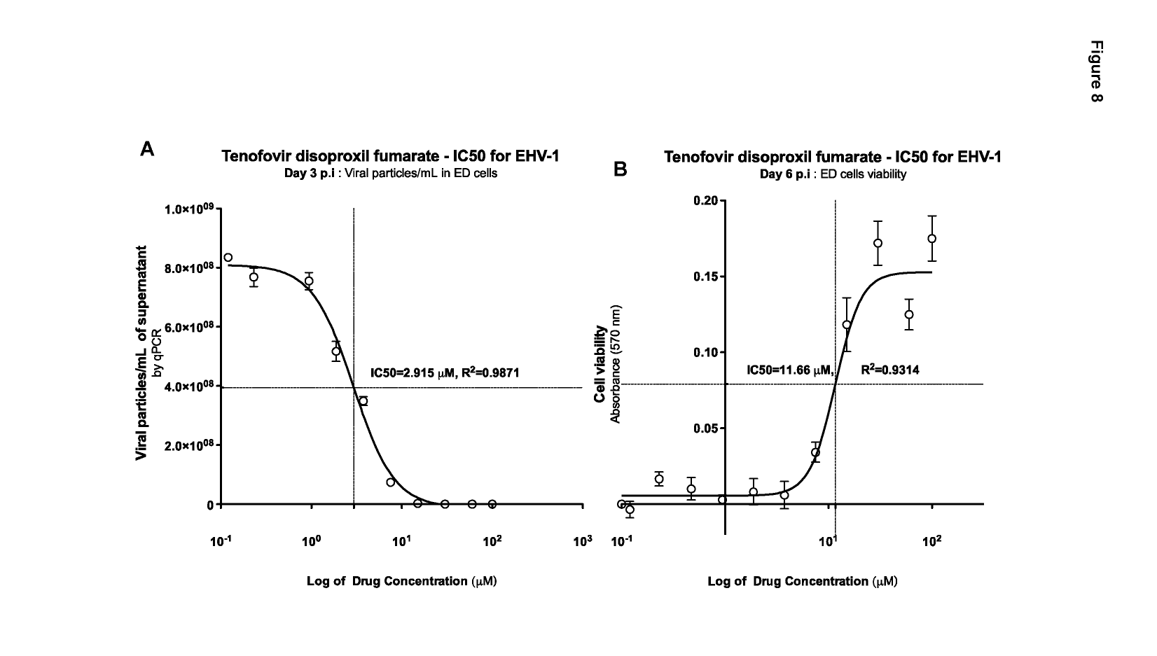

In MacF the average IC50 value obtained in three independent experiments, measured by qPCR at day 3 post infection, was 142.27 nM, with a standard deviation of 56.278. FIG. 7A show a representative experiment of IC50 values determined by qPCR at day 3 in MacF cells.

After 6 days post-infection reazurin-based cell viability assays (PrestoBlue, Thermofisher Scientific) were used to evaluate the increased cell numbers in the same conditions as indicated above. FIG. 7B show a representative experiment of the dose curve responses and IC₅₀ value determined at day 6 post infection by cell viability. The average IC₅₀ value determined in two independent experiments was 171.85 nM with a standard deviation of 28.63.

### Example 6: Tenofovir Disoproxil Fumarate Possess Antiviral Activity Against EHV-1

### Antiviral Activity Assay in Equine Dermal Cells

Antiviral activity of Tenofovir disoproxil fumarate was evaluated by measuring the reduction in number of EHV-1 viral particles in supernatant and by quantifying the upturn of viable cells numbers in infected cultures.

Equine Dermal cells (ATCC CCL57) (10⁵ cells/cm²) were seeded in 96 well plates 24 to 72 hours before infection in complete media and incubated in a humid chamber at 37° C. and 5% CO₂ until 95%-99% confluent monolayers were attained. Complete media was composed of DMEM medium (Gibco, Thermofisher Scientific) with 10% of Inactivated Fetal bovine serum (Gibco, Thermofisher Scientific), 1% of Glutamax (Gibco, Thermofisher Scientific) and 1% Penicilin-Streptamicin (Gibco, Thermofisher Scientific). Confluent cell monolayers were washed twice with HBSS to remove non-adherent cells, incubated with 150 μl of infection media (DMEM with 5% FBS) and pre-treated with different concentrations of Tenofovir disoproxil fumarate [from a stock of 100 mM in 100% DMSO] (Selleckchem, Germany). The pre-treatment with the drug preceded the infection.

ED cells were pre-treated with drug for 30 minutes before infection, and treatment maintained during infection.

To determine the IC₅₀ of Tenofovir disoproxil fumarate ten 1:2 serial dilutions ranging from 120 nM to 60 μM were performed in quintuplicates per each drug concentration. ED cells were pre-treated with drug for 30 minutes before infection, and treatment maintained during infection.

Cells were infected with EHV-1 at 1 MOI for 2 hours. Infective EHV-1 viral particles were produced in ED cells with an EHV-1 molecular clone purchased from ATCC (VR-2248). After infection cells were washed twice with HBSS to remove unbound virus and fresh complete media and fresh drug was replaced in each well. Treatment was maintained during 6 days without addition of fresh drug.

Quantification of EHV-1 viral particles was assayed 66 h or 72 h (3 days) post infection. Wells were screened for number of viral particles/mL of cell culture supernatant by using the EHV-1 specific quantitative Real Time PCR assay (CFX96 Biorad apparatus) as described above in example 5. The average IC₅₀ value obtained in four independent experiments, by means of qPCR at day 3 post infection, was 7.439 μM with a standard deviation of 7.015. FIG. 8A show a representative experiment of IC₅₀ values determined by qPCR at day 3 in ED cells. After 6 days post-infection reazurin-based cell viability assays (PrestoBlue, Thermofisher Scientific) were used to evaluate the increased cell numbers in the same conditions as indicated above in example 5. FIG. 8B show a representative experiment of the dose curve responses and IC₅₀ value determined at day 6 post infection by cell viability. The average IC₅₀ value determined in four independent experiments was 13.95 μM, with a standard deviation of 12.698.

### Tenofovir Disoproxil Fumarate CC₅₀ in ED Cells

Furthermore we addressed cytotoxicity of tenofovir disoproxil fumarate in ED cells. For that, ED cells (10⁵ cells/cm²) were seeded as mentioned above in 96-well plates. Confluent ED cell monolayers were washed twice with HBSS to remove non-adherent cells and pre-treated with tenofovir disoproxil fumarate [from a stock of 100 mM in 100% DMSO](Selleckchem, Germany) for 3 days. To determine drug CC₅₀, ten different 1:1.3 serial dilutions from 25.39 to 350 μM were performed in quintuplicates per each drug concentration. Cell viability was assessed by using the PrestoBlue reagent as mention above for IC₅₀ analysis and incubated for 24 hours.

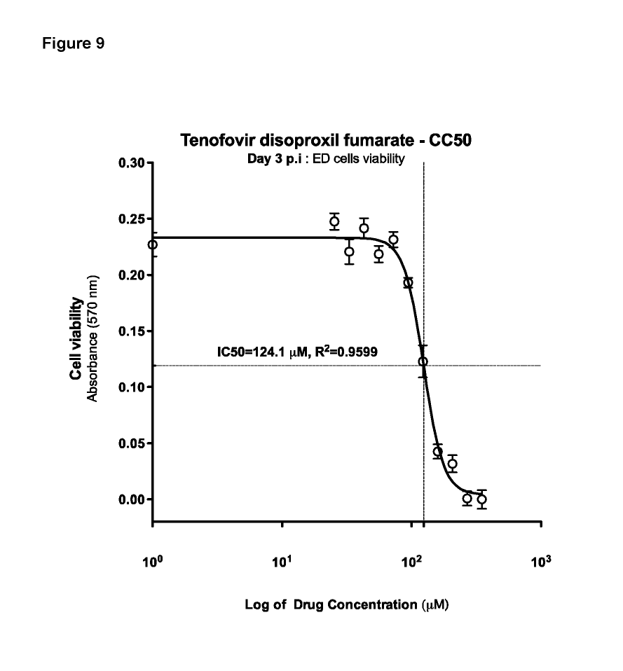

FIG. 9 shows the dose response curve of cell viability in the presence of different concentrations of tenofovir disoproxil fumarate. The results were analysed by Prism software by using the four-parameter function (log (drug) vs. response assuming a variable slope). The average results from two independent experiments showed that the CC₅₀ of the drug was 116.65 μM with a standard deviation of 10.535.

For EHV-1 the therapeutic index (CC₅₀/IC₅₀) retrieved for tenofovir disoproxil fumarate was of 8.334. The therapeutic index of tenofovir disoproxil fumarate was significantly lower to the therapeutic index of 1162.3 attained for adefovir dipivoxil.

### Antiviral Activity Assay in Equine Macrophage Like Cell Lines

Antiviral activity of Tenofovir disoproxil fumarate was also evaluated in macrophage like cell lines. MacF cells were seeded at a density of 10⁵ cells/cm2 in 96 well plates 24 to 72 hours before infection in complete media and incubated in a humid chamber at 37° C. and 5% CO2 until 95%-99% confluent monolayers were attained. Complete media was composed by DMEM medium (Gibco, Thermofisher Scientific) with 10% of Inactivated Fetal bovine serum (Gibco, Thermofisher Scientific), 2% of Glutamax (Gibco, Thermofisher Scientific) and 1% Penicilin-Streptamicin (Gibco, Thermofisher Scientific). Confluent cell monolayers were washed twice with HBSS to remove non-adherent cells, incubated with 150 μl of infection media (DMEM with 5% FBS) and pre-treated with different concentrations of Tenofovir disoproxil fumarate [from a stock of 100 mM in 100% DMSO] (Selleckchem, Germany). The pre-treatment with the drug preceded the infection.

MacF cells were pre-treated with drug for 30 minutes before infection, and treatment maintained during infection.

To determine the IC₅₀ of Tenofovir disoproxil fumarate ten 1:2 serial dilutions ranging from 120 nM to 60 μM were performed in quintuplicates per each drug concentration as described above for antiviral assay in ED cells.

EHV-1 cell infections, viral particle production and cell viability assays were assayed as described in example 5.

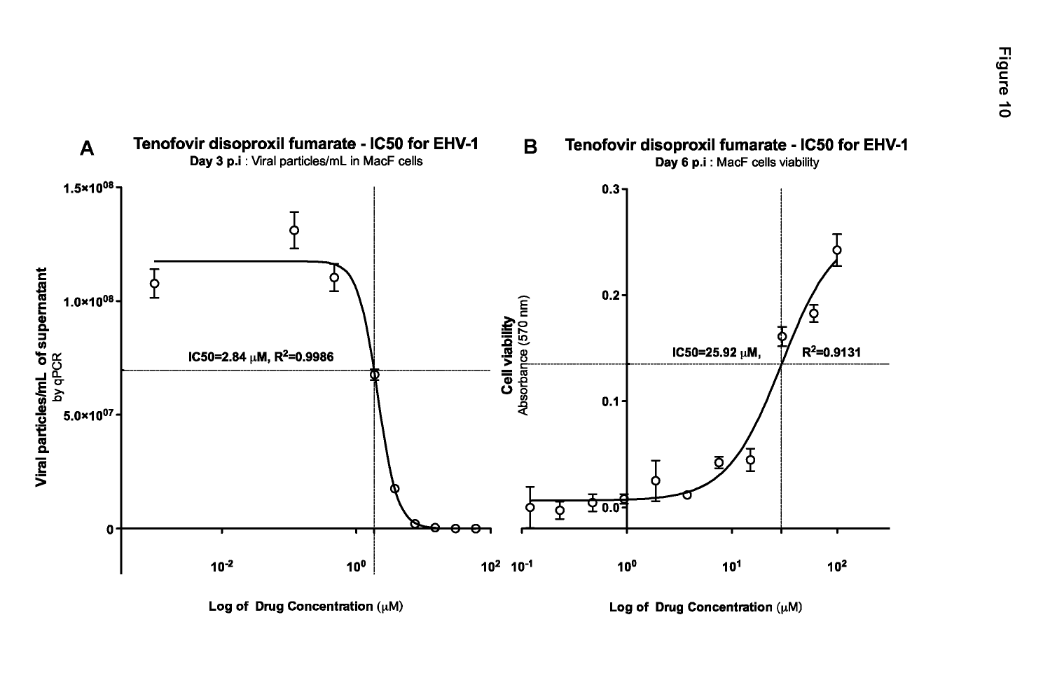

The average IC₅₀ value obtained in two independent experiments, by qPCR at day 3 post infection, was 1.13 μM with a standard deviation of 1.00. FIG. 10A show a representative dose response curve determined by qPCR at day 3 in MacF cells.

After 6 days post-infection reazurin-based cell viability assays (PrestoBlue, Thermofisher Scientific) were used to evaluate the increased cell numbers in the same conditions as indicated above in example 5. FIG. 10B show a representative experiment of the dose curve responses and IC₅₀ value determined at day 6 post infection by cell viability. The average IC₅₀ value determined in two independent experiments was 22.71 μM with a standard deviation of 9.63 All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearance of the phrases “in one embodiment” or “in an embodiment” in various places throughout this specification are not necessarily all referring to the same embodiment, but may do so. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

1.-21. (canceled)

22. A method of therapy of an equine viral infection and/or infection by an equine virus in an animal comprising administering to said animal a composition comprising at least one anti-viral compound, wherein said anti-viral compound is selected from adefovir or a pro-drug, equivalent or derivative thereof; and/or wherein said antiviral compound is selected from tenofovir or a pro-drug, equivalent or derivative thereof.

23. The method according to claim 22, wherein said anti-viral compound is selected from adefovir or a pro-drug, equivalent or derivative thereof; and/or tenofovir or a pro-drug, equivalent or derivative thereof.

24. The method according to claim 22 comprising administering to said animal an anti-viral compound of the Formula (I): 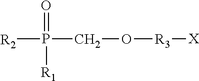
wherein
X is adenine, guanine, cytosine, thymine, uracil, 2,6-diaminopurine or hypoxanthine;
R₁ and R₂ are the same or different and are each independently selected from the group consisting of: OR₄, NH₂, NHR₄, NHR₅, NHR₄R₅, or N(R₅)₂; in some cases, R₁ and R₂ are linked with each other to form a cyclic group, in other cases, R₁ or R₂ is linked to R₃ to form a cyclic group;
R₃ represents C₁-C₂₀ alkyl which may be unsubstituted or substituted by substituents independently selected from the group consisting of hydroxy, oxygen, nitrogen and halogen; when R₃ is CH(CH₂OR₆)CH₂, R₁ and R₂ each independently represent OH, and R₆ is a hydrolyzable ester group;
R₄ represents hydrogen or a physiologically hydrolyzable group; R₄ may also be R₅′;
R₅ represents C₁-C₂₀ alkyl, alkoxy, amino, aryl or aryl-alkyl which may be substituted or unsubstituted by substitutents independently selected from the group consisting of hydroxyl, oxygen, nitrogen and halogen;
R₅′ represents C₄-C₂₀ alkyl, aryl or aryl-alkyl which may be substituted or unsubstituted by substitutents independently selected from the group consisting of hydroxyl, oxygen, nitrogen and halogen;
or a pharmaceutical or veterinary acceptable salt thereof.

25. The method according to claim 24, wherein said anti-viral compound is selected from adefovir and/or tenofovir.

26. The method according to claim 23, wherein said pro-drug, derivative or equivalent is selected from adefovir dipivoxil, tenofovir disoproxil, tenofovir disproxil fumarate and/or tenofovir alafenamide.

27. The method according to claim 22, wherein said equine viral infection is an equine lentiviral infection and/or wherein said equine virus is an equine lentivirus optionally wherein said equine lentiviral infection is equine infectious anaemia and/or wherein said equine lentivirus is equine infectious anaemia virus (EIAV).

28. (canceled)

29. The method according to claim 22, wherein said equine viral infection is an equine herpesviral infection and/or wherein said virus is an equine herpes virus optionally wherein said equine herpesvirus is selected from the group consisting of: EHV-1, EHV-2, EHV-3, EHV-4 and EHV-5.

30. (canceled)

31. The method according to claim 22, wherein said equine viral infection is a New Equine Viral infection and/or wherein said equine virus is New Equine Virus (NEV) optionally wherein said animal is an equine, optionally wherein said animal is a horse, further optionally wherein said animal is a NEV-seropositive horse.

32.-34. (canceled)

35. The method according to claim 24, wherein said physiologically hydrolyzable group is selected from the group consisting of CH₂C(O)N(R₅)₂, CH₂C(O)OR₅, CH₂OC(O)R₅, CH(R₅)OC(O)R₅ (R, S or RS stereochemistry), CH(R₅)C(O)R₅ (R, S or RS stereochemistry), CH_{z}C(R₅)₂CH₂OH or CH₂OR₅.

36. The method according to claim 24, wherein R₅ is selected from: tert-butyl and OCH(CH₃)₂.

37. The method according to claim 24, wherein said compound is stereoisomerically pure.

38. The method according to claim 22, wherein said composition is administered to said animal at least once weekly, preferably every 24 to 120 hours, preferably every 48 to 96 hours, preferably every 72 hours.

39. The method according to claim 22, to provide a total dose of 10-1000 mg/kg, during 1 to 6 weeks.

40. The method according to claim 22, wherein said composition is administered via a route selected from the group consisting of: oral, intravenous, intramuscular, subcutaneous, intranasal or intrapulmonary.

41. The method according to claim 22, wherein said therapy comprises alleviating one or more clinical symptoms of an equine viral infection optionally wherein said clinical symptoms are selected from the group consisting of: fever, thrombocytopenia, poor appetite, loss of body weight, wasting, anorexia, depression, malaise, apathy, lethargy, listlessness, weakness, weak pulse, irregular heartbeat, concurrent infections, low platelet count, anemia, edema, petechiation, hemorrhage, tachypneia, epistaxis (nosebleed), diarrhoea, blood-stained faeces, enlarged spleen, and swelling of the legs, abdomen, chest and/or genitals.

42.-46. (canceled)

47. A diagnostic method or method of screening for an equine virus, comprising:
(a) obtaining a sample from an animal;
(b) admixing with said sample a composition comprising at least one anti-viral compound;
(c) determining the presence or absence of an equine virus and/or equine viral particles and/or equine viral peptides and/or equine viral nucleic acids in said sample; optionally wherein said equine virus is resistant to an antiviral medicament; and/or wherein said equine virus is resistant to the anti-viral compound, and/or wherein said equine virus is resistant to a compound according to claim 22.

48.-50. (canceled)

51. A method for controlling a viral infection in a group of animals comprising the identification of a viral infection in an animal using the method of claim 47 and, optionally, the isolation of a equine virus-infected animal from other animals.

52. A pharmaceutical composition comprising at least one anti-viral compound for use in the method according to claim 22 and a pharmaceutically acceptable carrier, vehicle, diluent or excipient.

53. A kit comprising at least one anti-viral compound for use in the method according to claim 22 and optionally instructions for administration to said animal.

54. Use of a composition comprising at least one anti-viral compound for any of the following:
a) modulating reverse transcriptase activity in an equine virus,
b) inhibiting the replication of an equine virus in vitro,
c) promoting the survival of animal cell infected with an equine virus in vitro,
optionally wherein said animal cell is an equine cell, for example an equine dermal cell or equine macrophage.

55.-65. (canceled)
